(19) United States
(12) Patent Application Publication
(10) Pub. No.: US 2026/0234175 A1
(43) Pub. Date: Aug. 13, 2026

(54) PHOSPHINE LIGANDS AND THEIR USE IN COUPLING REACTIONS

(71) Applicants: BASF SE, Ludwigshafen (DE); THE REGENTS OF THE UNIVERSITY OF CALIFORNIA, Okland, CA (US)

(72) Inventors: Kyoungmin Choi, Berkeley, CA (US); John F. Hartwig, Berkeley, CA (US); Kailaskumar Borate, Navi Mumbai (IN); Carlos Lizandara Pueyo, Fremont, CA (US); Rahul Kaduskar, Navi Mumbai (IN); Harish Shinde, Navi Mumbai (IN); Roland Goetz, Ludwigshafen (DE); John Brunn, Fremont, CA (US)

(21) Appl. No.: 19/153,406

(22) PCT Filed: Feb. 9, 2024
(86) PCT No.: PCT/EP2024/053272

§ 371 Date: Aug. 4, 2025

Related U.S. Application Data
(60) Provisional application No. 63/484,067, filed on Feb. 9, 2023.

(30) Foreign Application Priority Data
Apr. 7, 2023 (EP) 23167193.4

(51) Int. Cl. C07F 9/6558; C07D 403/04

(52) U.S. Cl. CPC C07F 9/65583; C07D 403/04

(57) ABSTRACT

The present invention relates to phosphine ligands and the process for preparing such ligands. The present invention also relates the use and applications of these phosphine ligands in transition-metal catalyzed reactions.

### FIELD OF INVENTION

The present invention relates to phosphine ligands and the process for preparing such ligands. The present invention also relates the use and applications of these phosphine ligands in transition-metal catalyzed reactions.

### BACKGROUND

Complexes of transition metal catalysts are crucial in the production of pharmaceuticals, agrochemicals, and polymers, among other key applications in chemistry. The properties of both, the metal and the ligands attached to the metal atom, have an impact on the properties of the catalyst complexes. For instance, the structural characteristics of the ligands can affect the rate, regioselectivity, and stereoselectivity of reactions. For example, in many cases, bulky ligands exhibit low rate of an elementary step of oxidative addition. In coupling reactions, electron-withdrawing ligands increase the rate of reductive elimination from the metal center while decreasing the rate of oxidative addition, and electron-rich ligands increase the rate of oxidative addition to the metal center while decreasing the rate of reductive elimination from it.

Cross coupling reactions that are catalyzed by transition metals have drawn a lot of interest in organic synthesis to form carbon-carbon and/or carbon heteroatom bonds. Several types of well-known coupling reactions, such as the Heck reaction for the manufacture of olefins or dienes, the Buchwald-Hartwig amination reaction for the manufacture of amines, and the Suzuki-Miyaura reaction for the manufacture of diversified biaryls, have been used in pharmaceutical, material, and agricultural chemistry in recent years.

The structure of the ligand significantly affects the efficiency of the cross-coupling reactions. Thus, the design of ligands with appropriate steric and electronic properties, and with efficient synthetic protocols for their preparation is crucial to achieve cross-coupling reactions with specific combinations of reactants.

There are several prior art references which disclose transition-metal catalysts containing sterically bulky and electron-rich phosphines that are effective for the formation of aromatic carbon-carbon and carbon-nitrogen bonds.

However, there remains a need to develop improved ligands and reaction conditions for a variety of cross-coupling reactions.

The object of the present invention was to provide an improved ligand which can be used in a variety of cross-coupling reactions. Another object of the present invention was to satisfy the great need for novel, more productive & selective catalyst-ligand systems that yield products with high yield and high purity.

### SUMMARY OF THE INVENTION

These objects were achieved by the phosphine ligand of the present invention. It was surprisingly found that the phosphine ligand of the present invention was useful in Buchwald-Hartwig reactions, in which the process formed less or no unwanted product. Moreover, greater selectivity was obtained under normal reaction conditions, and with the use of cheaper raw materials.

One aspect of the present invention relates to the phosphine ligand of formula (I)

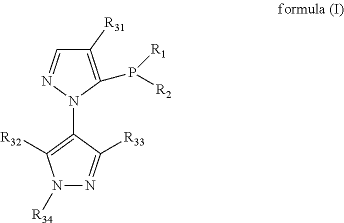

where,
R₁ and R₂ are, independently selected from substituted or unsubstituted C₃-C₁₄ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S,
R₃₁ is selected from substituted or unsubstituted, linear or branched C₁-C₆ alkyl, substituted or unsubstituted C₃-C₆ cycloalkyl, substituted or unsubstituted aryl and substituted or unsubstituted five or six membered heterocycloalkyl, where R₃₁ is in particular substituted or unsubstituted, linear or branched C₁-C₆ alkyl,
R₃₂, R₃₃, R₃₄, are each independently selected from substituted or unsubstituted, linear or branched C₁-C₆ alkyl, substituted or unsubstituted C₃-C₆ cycloalkyl, substituted or unsubstituted C₁-C₆ alkoxy, substituted or unsubstituted aryl, substituted or unsubstituted heteroaryl, substituted or unsubstituted N(R₁₂)₂, where R₁₂ is selected from substituted or unsubstituted C₁-C₆ alkyl, substituted or unsubstituted C₃-C₆ cycloalkyl, substituted or unsubstituted aryl, substituted or unsubstituted heteroaryl, substituted or unsubstituted heterocycloalkyl.

In another aspect, the present invention relates to the use of phosphine ligand of formula (I) in combination with transition metal catalyst to catalyze a reaction.

In another aspect the present invention relates to the use of catalysts comprising such ligands in various transition-metal-catalyzed carbon-heteroatom and carbon-carbon bond-forming reactions. The disclosed methods provide improvements in many features of the transition-metal-catalyzed reactions, including the range of suitable substrates, number of catalyst turnovers, reaction conditions, and efficiency.

A further aspect of the present invention also relates to the process of making the phosphine ligand of formula (I).

### DETAILED DESCRIPTION OF THE INVENTION

Before the present compositions and formulations of the presently claimed invention are described, it is to be understood that this invention is not limited to particular compositions and formulations described, since such compositions and formulation may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group, which preferably consists of these embodiments only. Furthermore, the terms ‘first’, ‘second’, ‘third’ or ‘a’, ‘b’, ‘c’, etc. and the like in the description and in the claims, are used for distinguishing between similar elements and not necessarily for describing a sequential or chronological order. It is to be understood that the terms so used are interchangeable under appropriate circumstances and that the embodiments of the presently claimed invention described herein are capable of operation in other sequences than described or illustrated herein. In case the terms ‘first’, ‘second’, ‘third’ or ‘(A)’, ‘(B)’ and ‘(C)’ or ‘(a)’, ‘(b)’, ‘(c)’, ‘(d)’, ‘i’, ‘il’ etc. relate to steps of a method or use or assay there is no time or time interval coherence between the steps, that is, the steps may be carried out simultaneously or there may be time intervals of seconds, min, hours, days, weeks, months or even years between such steps, unless otherwise indicated in the application as set forth herein above or below.

Furthermore, the ranges defined throughout the specification include the end values as well, i.e., a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment but may refer to the same embodiment. Further, as used in the following, the terms “preferably”, “more preferably”, “even more preferably”, “most preferably” and “in particular” or similar terms are used in conjunction with optional features, without restricting alternative possibilities. Thus, features introduced by these terms are optional features and are not intended to restrict the scope of the claims in any way.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any one of the claimed embodiments can be used in any combination.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

As used herein, “carbocycle” or “carbocyclic ring” is intended to include any stable monocyclic, bicyclic or tricyclic ring having the specified number of carbons, any of which may be saturated, unsaturated, or aromatic. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclo octane, [4.3.0]bicyclononane, [4.4.0]bicyclodecane and [2.2.2]bicyclooctane.

As used herein, “heterocycle” includes any ring structure (saturated or partially unsaturated) which contains at least one ring heteroatom (e.g., N, O or S). Examples of saturated heterocycles include, but are not limited to, morpholine, pyrrolidine, tetrahydrothiophene, piperidine, piperazine and tetrahydrofuran. Examples of unsaturated, unsaturated or heteroaromatic heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4H-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5 (4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl. Substituted with respect to a group in particular means that the respective group is partly or completely fluorinated, i.e. the hydrogen atoms are partly or completely replaced by fluorine atoms, or the group bears one or more radicals, in particular 1 to 5 radicals, which are selected from fluorine, chlorine, bromine, C₁-C₄ alkyl, fluorinated C₁-C₄ alkyl, C₁-C₄ alkoxy, fluorinated C₁-C₄ alkoxy, C₁-C₄ alkoxy-C₁-C₄-alkyl, C₃-C₆ cycloalkyl and phenyl.

“Alkyl” refers to a straight-chain or branched saturated hydrocarbon group. In certain embodiments, the alkyl group may have from 1-20 carbon atoms, in certain embodiments from 1-15 carbon atoms, in certain embodiments, 1-8 carbon atoms or 1 to 6 carbon atsm. The alkyl group may be unsubstituted. Alternatively, the alkyl group may be substituted. Unless otherwise specified, the alkyl group may be attached at any suitable carbon atom and, if substituted, may be substituted at any suitable atom. Typical alkyl groups include but are not limited to methyl, ethyl, n-propyl, iso-propyl, n-butyl, iso-butyl, sec-butyl, tert butyl, n-pentyl, n-hexyl and the like. Substituted with respect to the alkyl group in particular means that the alkyl group is partly or completely fluorinated or bears 1 or 2 radicals selected from C₁-C₄ alkoxy-

The term “cycloalkyl” is used to denote a saturated carbocyclic hydrocarbon radical. The cycloalkyl group may have a single ring or multiple condensed rings. In certain embodiments, the cycloalkyl group may have from 3 to 15 carbon atoms, in certain embodiments, from 3 to 10 carbon atoms, in certain embodiments, from 3 to 8 carbon atoms or from 3 to 6 carbon atoms. The cycloalkyl group may be unsubstituted. Alternatively, the cycloalkyl group may be substituted. Unless other specified, the cycloalkyl group may be attached at any suitable carbon atom and, if substituted, may be substituted at any suitable atom. Typical cycloalkyl groups include but are not limited to cyclopropyl, cyclobutyl, cyclopentyl, cyclohexyl, adamantyl and the like. Substituted with respect to the cycloalkyl group in particular means that the cycloalkyl group is partly or completely fluorinated or bears 1 to 4 radicals selected from C₁-C₄ alkyl and C₁-C₄ alkoxy.

“Alkoxy” and “cycloalkoxy” refer to an optionally substituted group of the formula alkyl-O— or cycloalkyl-O—, respectively, wherein alkyl and cycloalkyl are as defined above.

“Alkoxyalkyl” refers to an optionally substituted group of the formula alkoxy-alkyl-, wherein alkoxy and alkyl are as defined above.

“Aryl” refers to an aromatic carbocyclic group. The aryl group may have a single ring or multiple condensed rings. In certain embodiments, the aryl group can have from 6-20 carbon atoms, in certain embodiments from 6-15 carbon atoms, in certain embodiments, 6-12 carbon atoms. The aryl group may be unsubstituted. Alternatively, the aryl group may be substituted. Unless otherwise specified, the aryl group may be attached at any suitable carbon atom and, if substituted, may be substituted at any suitable atom. Examples of aryl groups include, but are not limited to, phenyl, naphthyl, anthracenyl and the like. In particular aryl is phenyl. Preferred aryl are phenyl and napththyl.

Substituted with respect to the aryl group in particular means that the aryl group bears 1 to 5 radicals selected from fluorine, chlorine, bromine, C₁-C₄ alkyl, fluorinated C₁-C₄ alkyl, C₁-C₄ alkoxy, fluorinated C₁-C₄ alkoxy, C₁-C₄ alkoxy-C₁-C₄-alkyl, C₃-C₆ cycloalkyl and phenyl.

“Heterocycloalkyl” means a substituent derived from a saturated heterocycle and which has preferably 1 or 2 heteroatoms selected from O, N and S as ring members. Preferred is 5- or 6-membered heterocyclcoalkyl. Examples include pyrrolidinyl, piperidinyl, piperazinyl, morpholinyl, tetrahydrofuryl and tetrahydrothiophenyl.

Substituted with respect to the heterocycloalkyl in particular means that the heterocycloalkyl group bears 1 to 5 radicals selected from C₁-C₄ alkyl, fluorinated C₁-C₄ alkyl, C₁-C₄ alkoxy, fluorinated C₁-C₄ alkoxy, C₁-C₄ alkoxy-C₁-C₄-alkyl, C₃-C₆ cycloalkyl and phenyl.

“Hetaryl” means a substituent derived from an aromatic heterocycle and which has preferably 1 or 2 heteroatoms selected from O, N and S as ring members. Preferred is 5- or 6-membered hetaryl and 5- or 6-membered hetaryl fused to a benzene ring. Substituted with respect to the hetaryl group in particular means that the hetaryl group bears 1 to 5 radicals selected from fluorine, chlorine, bromine, C₁-C₄ alkyl, fluorinated C₁-C₄ alkyl, C₁-C₄ alkoxy, fluorinated C₁-C₄ alkoxy, C₁-C₄ alkoxy-C₁-C₄-alkyl, C₃-C₆ cycloalkyl and phenyl.

“Coupling” refers to a chemical reaction in which two molecules or parts of a molecule join together (Oxford Dictionary of Chemistry, Sixth Edition, 2008).

With respect to formulae the abbreviation “Ph” means phenyl and the abbreviation “Ad” means 1-adamantyl.

In an embodiment, the present invention relates to a phosphine ligand of formula (I)

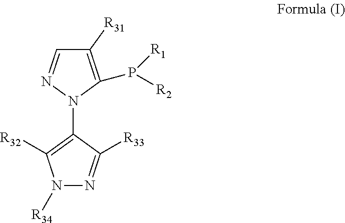

where,
R₁ and R₂ are, independently selected from substituted or unsubstituted C₃-C₁₄ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S,
R₃₁ is selected from substituted or unsubstituted, linear or branched C₁-C₆ alkyl, such as methyl, ethyl, n-propyl, 2-propyl, n-butyl, 2-butyl or isobutyl, substituted or unsubstituted C₃-C₆ cycloalkyl, such as cyclopentyl or cyclohexyl, substituted or unsubstituted aryl, such as phenyl, and substituted or unsubstituted five or six membered heterocycloalkyl, in particular N-pyrrolidinyl, N-morpholinyl or N-piperidinyl, where R₃₁ is in particular substituted or unsubstituted, linear or branched C₁-C₆ alkyl;
R₃₂, R₃₃, R₃₄, are each independently selected from substituted or unsubstituted, linear or branched C₁-C₆ alkyl, substituted or unsubstituted C₃-C₆ cycloalkyl, substituted or unsubstituted C₁-C₆ alkoxy, substituted or unsubstituted aryl, substituted or unsubstituted heteroaryl, substituted or unsubstituted N(R₁₂)₂, where R₁₂ is selected from substituted or unsubstituted C₁-C₆ alkyl, substituted or unsubstituted C₃-C₆ cycloalkyl, substituted or unsubstituted aryl, substituted or unsubstituted heteroaryl, substituted or unsubstituted heterocycloalkyl.

In particular R₁ and R₂ are independently selected from cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl, tetrahydronaphthyl, [3.3.0]bicyclo octane, [4.3.0]bicyclononane, [4.4.0]bicyclodecane and [2.2.2]bicyclooctane.

Preferably R₁ and R₂ are independently selected from adamantyl, cyclohexyl and cyclopentyl. More preferably, R₁ and R₂ are independently selected from adamantyl and cyclohexyl.

In particular, R₃₁ is selected from substituted or unsubstituted C₁-C₆ alkyl, such as methyl, ethyl, n-propyl, isopropyl, n-butyl, 2-butyl, isobutyl, n-pentyl, isopentyl, n-hexyl and pentafluoroethyl. In particular, R₃₁ may also be a cyclic radical selected from cyclopropyl, cyclobutyl, cyclopentyl, phenyl, or 1-piperidinyl. More preferably, R₃₁ is unsubstituted C₁-C₆ alkyl. Even more preferably, R₃₁ is selected from methyl, ethyl, n-propyl, isopropyl and n-butyl. Especially, R₃₁ is selected from methyl, iso propyl and n-propyl. R₃₁ may also be selected from phenyl, optionally substituted by 1, 2, or 3 substituents selected from halogen, C₁-C₆ alkyl and C₁-C₆ alkoxy, and N-heterocyclyl, such as N-pyrrolidinyl, N-morpholinyl or N-piperidinyl.

In particular, R₃₂, R₃₃, R₃₄, are, independently selected from substituted or unsubstituted straight chain C₁-C₆ alkyl, substituted or unsubstituted branched chain C₃-C₆ alkyl, substituted or unsubstituted C₃-C₆ cycloalkyl, substituted or unsubstituted C₁-C₆ alkoxy, substituted or unsubstituted aryl or substituted and unsubstituted heteroaryl. Preferably R₃₂, R₃₃, R₃₄ are aryl, in particular phenyl.

In a preferred embodiment, the phosphine ligand according to present invention is selected from the compound of the following formulae:

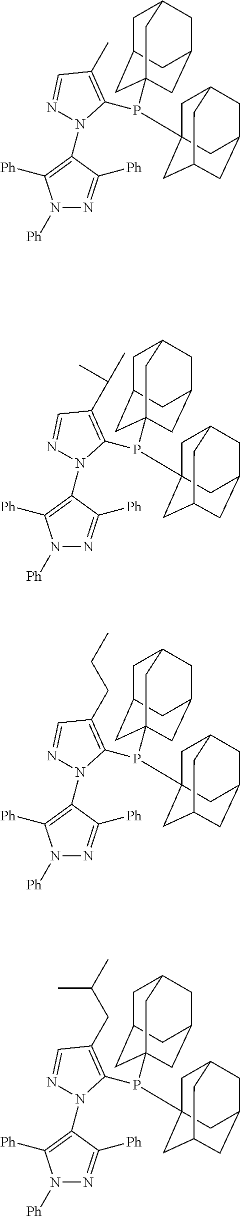

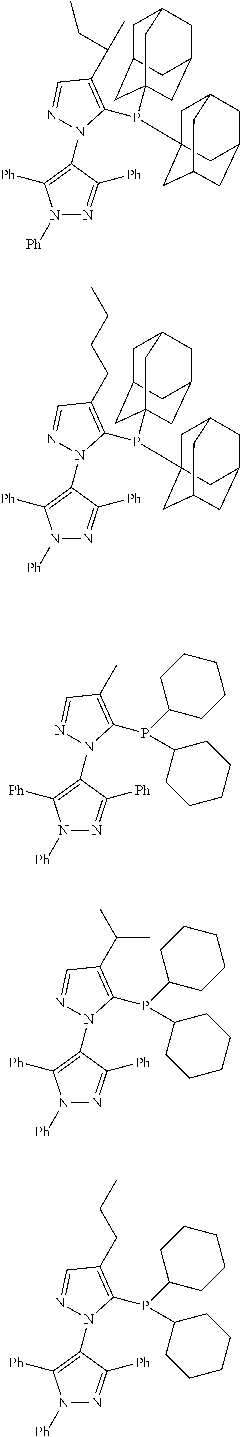

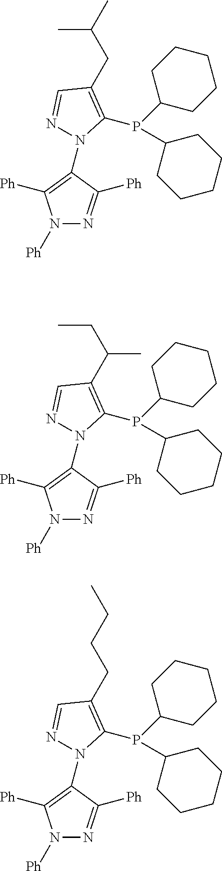

where Ph means phenyl.

### Process to Synthesize the Ligand

In one further aspect the present invention provides a method of synthesizing the phosphine ligand of formula (I), comprising at least the steps of,
a) Lithiation of compound of formula (II)

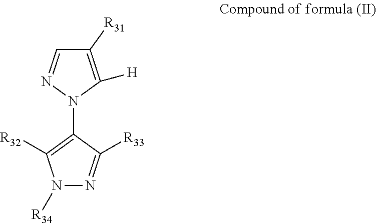

where,
R₃₁ is as defined for formula (I)
R₃₂, R₃₃, R₃₄, are each independently as defined for formula (I),
b) reacting the lithiated compound of formula (II) with compound of formula (III)

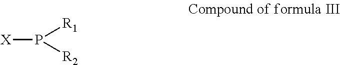

where X is Cl or Br,
R₁ and R₂ as defined from formula (I),
to form phosphine ligand of formula (I) wherein the phosphine ligand of formula (I) is in a crude form,
c) optionally purifying the phosphine ligand of formula (I) obtained in step b).

In one embodiment, the present invention further provides a method of synthesizing the phosphine ligand of formula (I), comprising at least the steps of,
a) Lithiation of compound of formula (II)
   where
   R₃₁ is as defined for formula (I)
   R₃₂, R₃₃, R₃₄, are each independently as defined for formula (I),
b) reacting the lithiated compound of formula (II) with a compound of formula (III) where X is Cl or Br,
   R₁ and R₂ are each independently as defined for formula (I), to form phosphine ligand of formula (I) wherein the phosphine ligand of formula (I) is in a crude form,
c) optionally purifying the phosphine ligand of formula (I) obtained in step b) by salt formation with an acid,
d) optionally further purifying the phosphine ligand of formula (I) in step c) to give phosphine ligand of formula (I) in the pure form.

In step a), the lithiation of the compound of formula (II) is generally carried out in by a lithiating reagent, in particular an organolithium reagent. In particular, the lithiation in step a) is carried out in the presence of organolithium reagents selected from methyl lithium, ethyl lithium, t-butyl lithium, n-butyl lithium, sec-butyl lithium, iso-propyl lithium, lithium di-isopropyl amide or cyclohexanyl lithium; Preferred lithiating agents n-butyllithium or lithium diisopropylamide.

The lithiation of step a) is typically carried out in an inert organic solvent. The inert organic solvent is typically an aprotic organic solvent or a mixture of aprotic organic solvents.

In step a), the organic solvent is preferably selected from the group of aliphatic and alicyclic ethers, such as tetrahydrofuran, 2-methyl tetrahydrofuran, diethyl ether, aliphatic, cycloaliphatic or aromatic hydrocarbons, such as hexanes, heptane, cyclohexane, toluene, or xylenes, or a combination thereof.

In particular, the lithiation of step a) is carried out at a temperature in the range of −80° C. to −50° C., preferably in the range of −80° C. to −60° C., more preferably in the range of −80° C. to −70° C. for period of 1 hour to 5 hours, preferably 1 hour to 4 hours, more preferably 1 hour to 2 hours.

In particular, the lithiation in step a) is carried out such that the molar ratio of compound of formula II to the organolithium reagent is in the range of 0.8 moles to 2 moles per 1 mole of the organolithium reagent.

In step b) the lithiated compound of formula (II) is reacted with compound of formula (III) to form phosphine ligand of formula (I) wherein the phosphine ligand of formula (I) is in a crude form.

In particular, the reaction of step b) is carried out at a temperature in the range of −20° C. to −2° C., preferably in the range of −15° C. to −3° C., more preferably in the range of −12° C. to −3° C. The reaction of step b) is in particular carried out for a period of 1 hour to 5 hours, preferably 1 hour to 4 hours, more preferably 1 hour to 2 hours.

In an embodiment in step b), wherein the reaction is carried out in the presence of a solvent selected from the solvent is selected from tetrahydrofuran, 2-methyl tetrahydrofuran, diethyl ether, hexane, toluene, or a combination thereof.

In an embodiment, in step b), the reaction is carried out wherein the molar ratio of compound of formula III to the lithiated compound of formula (II) is in the range of 0.8 mol to 2 mol equivalent.

In one embodiment, the method for synthesizing the phosphine ligand of formula (I) is a one-pot, or a two-step process.

In one embodiment, the method for synthesizing the phosphine ligand of formula (I) further comprises a step c) of purifying said phosphine ligand by salt formation with an acid.

In an embodiment, in step c) the acid is selected from hydrochloric acid, sulfuric acid, methane sulfonic acid, p-toluene sulfonic acid, tetra fluoroboric acid, preferably tetrafluoro boric acid.

In an embodiment, in step d) optionally the salt form of the phosphine ligand of formula (I) of step c) is further purified to obtain the phosphine ligand of formula (I) in the pure form.

In an embodiment, in step d), the pure form of the phosphine ligand of formula (I) is obtained by adding a base selected from sodium hydroxide, potassium hydroxide, lithium hydroxide, sodium carbonate, potassium carbonate, cesium carbonate, sodium bicarbonate, potassium bicarbonate, cesium bicarbonate, sodium methoxide, potassium methoxide and heating the reaction mixture for a period of 2-6 hours and further performing routine reaction work-up to obtain pure phosphine ligand of formula (I).

In one embodiment, the phosphine ligand of formula (I) synthesized by the methods of the present invention is in particular selected from the group consisting of:

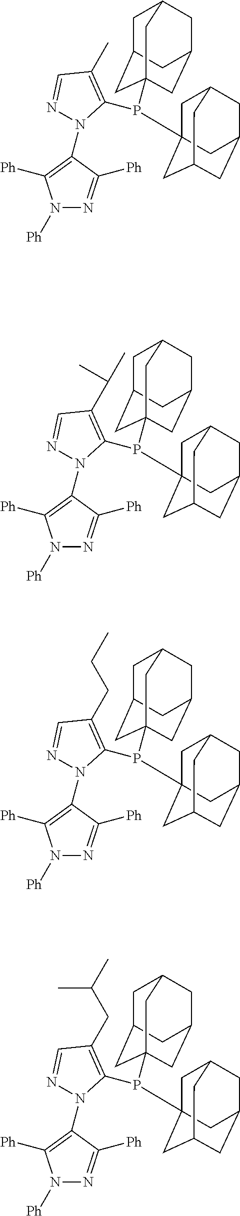 [SMILES: Cc1cnn(-c2c(-c3ccccc3)nn(-c3ccccc3)c2-c2ccccc2)c1P(C12CC3CC(CC(C3)C1)C2)C12CC3CC(CC(C3)C1)C2 ; CC(C)c1cnn(-c2c(-c3ccccc3)nn(-c3ccccc3)c2-c2ccccc2)c1P(C12CC3CC(CC(C3)C1)C2)C12CC3CC(CC(C3)C1)C2 ; CCCc1cnn(-c2c(-c3ccccc3)nn(-c3ccccc3)c2-c2ccccc2)c1P(C12CC3CC(CC(C3)C1)C2)C12CC3CC(CC(C3)C1)C2 ; CC(C)Cc1cnn(-c2c(-c3ccccc3)nn(-c3ccccc3)c2-c2ccccc2)c1P(C12CC3CC(CC(C3)C1)C2)C12CC3CC(CC(C3)C1)C2]

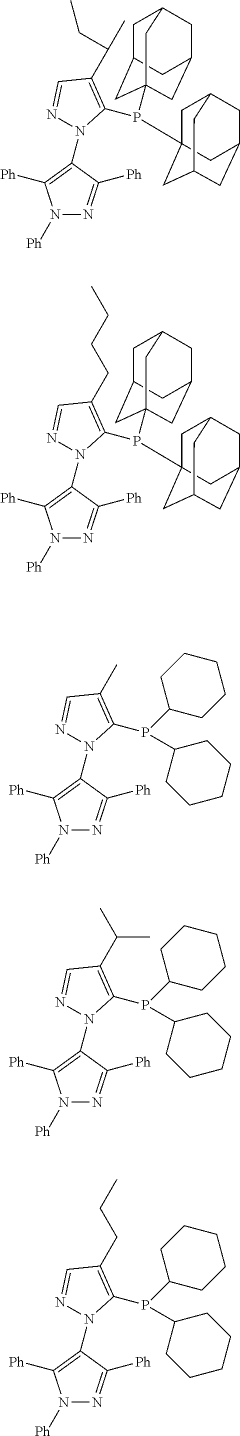 [SMILES: CCC(C)c1cnn(-c2c(-c3ccccc3)nn(-c3ccccc3)c2-c2ccccc2)c1P(C12CC3CC(CC(C3)C1)C2)C12CC3CC(CC(C3)C1)C2 ; CCCCc1cnn(-c2c(-c3ccccc3)nn(-c3ccccc3)c2-c2ccccc2)c1P(C12CC3CC(CC(C3)C1)C2)C12CC3CC(CC(C3)C1)C2 ; Cc1cnn(-c2c(-c3ccccc3)nn(-c3ccccc3)c2-c2ccccc2)c1P(C1CCCCC1)C1CCCCC1 ; CC(C)c1cnn(-c2c(-c3ccccc3)nn(-c3ccccc3)c2-c2ccccc2)c1P(C1CCCCC1)C1CCCCC1 ; CCCc1cnn(-c2c(-c3ccccc3)nn(-c3ccccc3)c2-c2ccccc2)c1P(C1CCCCC1)C1CCCCC1]

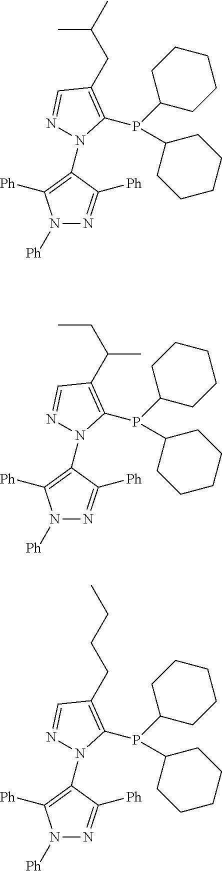 [SMILES: CC(C)Cc1cnn(-c2c(-c3ccccc3)nn(-c3ccccc3)c2-c2ccccc2)c1P(C1CCCCC1)C1CCCCC1 ; CCC(C)c1cnn(-c2c(-c3ccccc3)nn(-c3ccccc3)c2-c2ccccc2)c1P(C1CCCCC1)C1CCCCC1 ; CCCCc1cnn(-c2c(-c3ccccc3)nn(-c3ccccc3)c2-c2ccccc2)c1P(C1CCCCC1)C1CCCCC1]

The compounds of the formula (II) and (III) are commercially available or can be obtained by standard procedures of organic chemistry.

For example the compounds of the formula (II) can be obtained by a process comprising
i) reacting a pyrazole compound of the formula (IV) with a 1,3 diketone of the formula (V) in the presence of a halogenating agent;

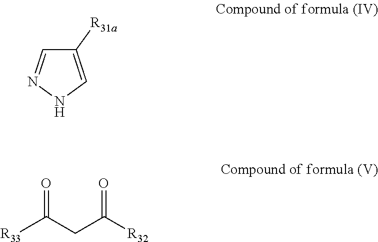

ii) reaction of the obtained intermediate with a hydrazine compound of the formula (VI)

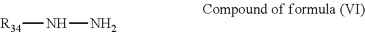

where R₃₂, R₃₃ and R₃₄ in formulae (V) and (VI) are as defined in the context of the formula (I) and where R₃₁ₐ in formulae (IV) has one of the meanings given for R₃₁ or is halogen, such as chlorine bromine or iodine.

The halogenating agent in step i) is typically a cyclic N-haloimide of a dicarboxylic acid, such as N-bromo succinimide or N-chloro succinimide.

The reaction of step i) is typically carried out in an inert organic solvent, such as N-alkylamides, in particular N—C₁-C₄ alkyl lactams, e.g. N-methylpyrrolidone, halogenoalkanes, in particular chloro-C₁-C₄ alkanes, such as dichloromethane or dichloroethane or mixtures thereof.

The reaction of step ii) is typically carried out in a protic organic solvent. Suitable protic organic solvents are in particular aliphatic carboxylic acids, in particular C₁-C₄ alkanoic acids, such as acetic acid, propionic acid and the like and mixtures thereof with alkanols, in particular C₁-C₄ alkanols, such as methanol or ethanol.

If R₃₁ₐ is halogen, the preparation of the compound of the formula (II) typically comprises a further step iii), where the halogen atom is replaced by a suitable nucleophilic compound, e.g. by a cyclic amine or by means of an organo zinc compound, optionally in the presence of a palladium catalyst, e.g. by analogy to a Negeshi type coupling, or an organo copper compound.

According to the invention, the phosphine ligands of formula (I) are used as catalysts in combination with transition metal complexes or transition metal salts of subgroup VIII of the Periodic Table of the Elements, for example palladium, nickel, platinum, rhodium, iridium, ruthenium or cobalt. As a rule, the phosphine ligands according to the invention can be added in situ to appropriate transition metal precursor compounds and used in this form for catalytic applications.

The transition metal compounds used are preferably palladium or nickel compounds and particularly preferably palladium compounds.

It may be advantageous on occasion to prepare defined mono-, di-, tri- or tetra phosphine complexes of said transition metals first and then use these for catalytic reactions.

It is preferable to use palladium and nickel catalysts containing the phosphine ligand of formula (I) according to the invention.

It is particularly preferable to use palladium catalysts containing the ligands according to the invention. The ligands according to the invention are normally added in situ to palladium (II) salts or to palladium (II) or palladium (0) complexes. However, it may be advantageous to prepare palladium (0)- or palladium (II)-phosphine complexes of the phosphines according to the invention direct and then use these for catalytic applications. This increases the initial catalyst activity in some instances.

Examples of palladium components that can be used with the ligands according to the invention are palladium (II) acetate, palladium (II) chloride, palladium (II) bromide, lithium tetrachloropalladate (II), palladium (II) acetylacetonate, palladium (0)-benzylideneacetone complexes, palladium (0) tetrakis(triphenylphosphine), palladium (0) bis(tri-o-tolylphosphine), palladium (II) propionate, palladium (II) bis(triphenylphosphine)dichloride, palladium (0)-diallyl ether complexes, palladium (II) nitrate, palladium (II) chloride. bis(acetonitrile), palladium (II) chloride bis(benzonitrile) and other palladium (0) and palladium (II) complexes.

Generally, for catalytic applications, the phosphine ligand is used in excess relative to the transition metal. The ratio of transition metal to ligand is preferably from 1:1 to 1:1000. Ratios of transition metal to ligand of 1:1 to 1:100 are particularly preferred. The exact transition metal/ligand ratio to be used depends on the specific application and also on the amount of catalyst used. Thus, in general, it is conventional to use low transition metal/ligand ratios in the case of very low transition metal concentrations (<0.01 mol %) than in the case of transition metal concentrations of between 0.5 and 0.01 mol % of transition metal.

The phosphine ligands of formula (I) are thermally very stable. It is thus possible to use the catalysts according to the invention at reaction temperatures of up to 250° C. or more. The catalysts are preferably used at temperatures of 20 to 200° C.; it has proved advantageous in many cases to work at temperatures of 30 to 180° C., preferably of 40 to 160° C. The ligands can also be used in pressure reactions without loss of activity, the operating pressure conventionally being up to only 100 bar, but preferably in the normal pressure range of up to 60 bar.

The phosphine ligand of formula (I) prepared according to the invention have proved particularly advantageous as ligand components for the catalytic preparation of arylated olefins (Heck reactions), biaryls (Suzuki reactions), α-aryl ketones and amines from aryl halides. However, it is obvious to those skilled in the art that other transition metal-catalyzed reactions, such as the metathesis or hydrogenation of double bonds or carbonyl compounds, especially however palladium-catalyzed and nickel-catalyzed carbonylation's of aryl halides, alkynylations with alkynes (Sonogashira couplings) and cross couplings with metal-organic reagents (zinc reagents, tin reagents, etc.), can also be catalyzed with the novel catalyst systems.

The catalysts of the present invention may also be used for carbon-heteroatom coupling reactions, such as carbon-nitrogen coupling reactions (i.e., Buchwald-Hartwig reaction), or carbon-oxygen or carbon-sulfur coupling reactions.

One particular advantage of the ligands according to the invention is the high activity which the ligands induce in the activation of cost-effective chloroaromatics. As shown in the experimental Examples, palladium catalysts with the adamantly phosphines are significantly superior to the best existing catalyst systems of Buchwald (J. P. Wolfe, S. L. Buchwald, Angew. Chem. 1999, 111, 2570; Angew. Chem. Int. Ed. Engl. 1999, 38, 2413) and Fu (A. F. Littke, G. C. Fu, Angew. Chem. 1998, 110, 3586; Angew. Chem. Int. Ed. Engl. 1998, 37, 3387). Thus, with the catalyst systems according to the invention, it is even possible to achieve turnover numbers in the order of >10,000 with chloroaromatics as substrates and TONs of >500,000 with bromoaromatics as starting materials, making the described catalyst and ligand systems useful for large-scale industrial purposes.

The properties of the adamantly phosphines are particularly surprising. Although adamantyl radicals have been known for a long time in organic chemistry, no importance has been attached to phosphine ligands containing adamantyl groups. Consequently, alkyladamantyl phosphines have not hitherto been described for catalytic applications. It was surprising to find that, in certain catalytic applications, adamantyl ligands are significantly superior to all other known phosphine ligands.

The phosphine ligand of formula (I) prepared according to the invention can be used for the preparation of aryl olefins, dienes, diaryls, benzoic acid derivatives, acrylic acid derivatives, aryl alkanes, alkynes and amines. The compounds prepared in this way can be used inter alia as UV absorbers, intermediates for pharmaceuticals and agrochemicals, ligand precursors for metallocene catalysts, perfumes, active substances, and structural units for polymers.

In an embodiment, the present invention is directed to the use of the phosphine ligand of formula (I) to catalyze a reaction.

In another embodiment, the present invention is directed to a method for catalyzing a reaction in presence of a phosphine ligand of formula (I).

In an embodiment, the present invention is directed to the use of the phosphine ligand of formula (I) along with at least one metal catalyst to catalyze a reaction.

In another embodiment, the present invention is directed to a method for catalyzing a reaction in presence of a phosphine ligand of formula (I) and at least one metal catalyst.

In an embodiment, the metal catalyst is transition metal catalyst.

In an embodiment, the use or method of the phosphine ligand of formula (I) to catalyzing a reaction wherein the reaction is selected from carbon-carbon coupling, carbon-heteroatom coupling, or polymerization reaction.

In an embodiment, the use or method of the phosphine ligand of formula (I) to catalyzing a reaction wherein the reaction is selected from Heck reaction, Suzuki reaction, Sonogashira reaction, Negishi reaction, ketone alpha-arylation, aldehyde alpha-arylation, allylic substitution, Buchwald-Hartwig reaction, carbon-oxygen coupling or carbon-sulfur coupling.

In an embodiment, the phosphine ligand of formula (I) according to the present invention is used in Buchwald-Hartwig reaction.

In an embodiment, the use or method of the phosphine ligand of formula (I) to catalyzing a reaction wherein the reaction is the Buchwald-Hartwig reaction to form aryl amines comprising at least the step of:
A) amination of aryl halide in the presence of
   i) at least one base,
   ii) at least one metal catalyst, in particular a transition metal catalyst comprising a transition metal of subgroup VIII of the Periodic Table of the Elements, for example palladium, nickel, platinum, rhodium, iridium, ruthenium or cobalt, especially a transition metal selected from palladium and nickel, and especially a transition metal catalyst comprising palladium, and
   iii) at least one ligand of formula (I).

In an embodiment, the phosphine ligand of formula (I) according to the present invention is used in the preparation of substituted or unsubstituted aryl amines comprising at least the step of:
i) amination of substituted or unsubstituted aryl halide in the presence of source of ammonia, at least one base, at least one metal catalyst, in particular a transition metal catalyst comprising a transition metal of subgroup VIII of the Periodic Table of the Elements, for example palladium, nickel, platinum, rhodium, iridium, ruthenium or cobalt, more particularly a transition metal selected from palladium and nickel, and especially a transition metal catalyst comprising palladium.

In an embodiment the aryl halide has the formula

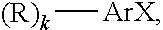

where X is halogen selected from chloride, bromide and iodide,
Ar is aryl group selected from simple aromatic rings (single or polycyclic) such as benzene, naphthalene, anthracene and phenanthrene; or heteroaromatic rings (single or polycyclic), such as pyrrole, thiophene, thianthrene, furan, pyran, isobenzofuran, chromene, Xanthene, phenoxathiin, pyrrole, imidazole, pyrazole, thiazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, perimidine, phenanthroline, phenazine, phenarsazine, phenothiazine, furazan, phenoxazine, pyrrolidine, oxolane, thiolane, oxazole, piperidine, piperazine, morpholine and the like;
k is 0 or an integer different from 0, e.g. 1, 2, 3, 4 or 5;
(R)ₖ represents one or more substituents on the aryl group, which are independently selected from, halogen, C₁-C₄-alkyl, C₂-C₄-alkenyl, C₁-C₄-halogenalkyl, —CN, —NO₂, —NH₂, phenyl, 5- or 6-membered heteroaryl, —O—C₁-C₄-alkyl, —O—C₁-C₄-halogenalkyl, tri-C₁-C₃-alkylsilyl, carbonyl C₁-C₃-alkyl, amido, such as —C(═O)—NR^{a}R^{b}, sulfonamide such as —S(═O)₂—NR^{a}R^{b}, sulfonyl, such as SO₂—C₁-C₃-alkyl or SO₂-aryl, phenyl sulfonyl or tolylsulfonyl, or —S—C₁-C₃ alkyl, where R^{a} and R^{b}, on each occurrence are independently of each other selected from hydrogen, C₁-C₄ alkyl and phenyl.

In an embodiment, X is chloride.

In another embodiment, X is bromide.

In another embodiment Ar is phenyl, X is chloride or bromide and R is selected from,

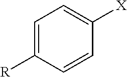

| R | X | R | X |
|---|---|---|---|
| —N(CH₃)₂ | Cl | —C(═O)-t-C₄H₉ | Cl |
| —O(CH₃) | Cl | —C(═O)—CH₃ | Cl |
| -TMS¹⁾ | Cl | —C(═O)—N(C₂H₅)₂ | Cl |
| -TMS¹⁾ | Br | —C(—OH)—CH₃ | Cl |
| -nC₄H₉ | Cl | —NH₂ | Cl |
| —C₆H₅ | Cl | —CN | Cl |
| —F | Cl | —NO₂ | Cl |
| —F | Br | —C(═CH₂)—CH₃ | Cl |
| —O(CF₃) | Cl | —S(CH₃) | Cl |
| —CF₃ | Cl | —S(O₂)—C₆H₅ | Cl |
| —S(O₂)—N(CH₃)₂ | Br | | |

¹⁾TMS = trimethyl silyl

In another embodiment, X is chloride or bromide and R—Ar is selected from

R-Ar X

| R-Ar | X | R-Ar | X |
|---|---|---|---|
| 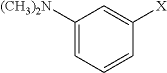 | Br | 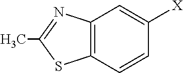 | Cl |
| 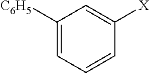 | Br | 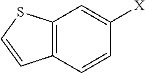 | Br |
| 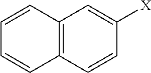 | Cl | 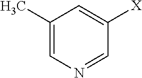 | Cl |
| 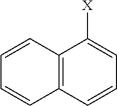 | Cl | 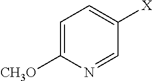 | Cl |
| 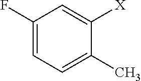 | Cl | 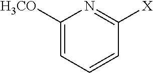 | Cl |
| 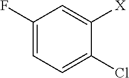 | Cl | 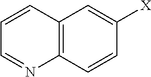 | Cl |
| 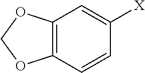 | Cl | 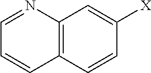 | Cl |
| 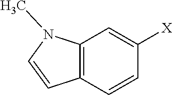 | Br | 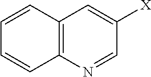 | Cl |
| 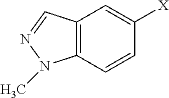 | Br | 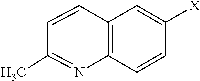 | Cl |
| 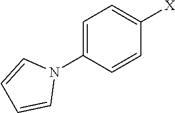 | Cl | 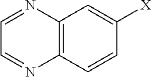 | Cl |

In an embodiment, R is halogen, preferably fluoro, Ar is phenyl, and X is chloride.

In an embodiment, the phosphine ligand of formula (I) according to the present invention is used in the preparation of a mono, di-, tri-, or tetrafluoro aryl amine comprising at least the step of:
A) amination of a mono, di-, tri-, or tetra fluoroaryl chloride in the presence of source of ammonia, at least one base, at least one metal catalyst complex, in particular a transition metal catalyst comprising a transition metal of subgroup VIII of the Periodic Table of the Elements, for example palladium, nickel, platinum, rhodium, iridium, ruthenium or cobalt, especially a transition metal selected from palladium and nickel, and especially a transition metal catalyst comprising palladium.

In an embodiment, the mono, di-, tri- or tetra fluoroaryl chloride is selected from the group consisting of mono flurochlorobenzene, 1,2-fluorochlorobenzene; 1,3-fluorochlorobenzene; 3,4-difluorochlorobenzene; 2,5-difluorochlorobenzene; 2,3-difluorochlorobenzene; 3,4-trifluorochlorobenzene, 2,3,4-trifluoro chlorobenzene and 2,3,5,6-tetra-fluorochlorobenzene.

In an embodiment of the presently claimed invention, ammonia can used in the form of a solution in at least one organic solvent or in gaseous form.

If the ammonia is dissolved in at least one organic solvent before mixing with the other components, i.e., the starting material, the at least one base, the at least one catalyst complex, the at least one ligand and the at least one organic solvent, then the at least organic solvent for dissolving the ammonia is preferably identical to the at least one organic solvent for conducting the reaction.

In yet another embodiment, the Buchwald-Hartwig reaction is carried out in the presence of at least one organic solvent selected from the group of cyclic ethers, acyclic aliphatic ethers and aliphatic alcohols wherein the solvent is selected from the group consisting of 1,4-dioxane, 1,3-dioxane, tetrahydrofuran, cyclopentyl methyl ether, tert-butyl-methyl-ether, methanol and isopropanol, preferably the solvent is 1,4-dioxane or 1,3-dioxane.

In an embodiment, the at least one base is selected from the group consisting of alkoxides, carbonates, bicarbonates, hydroxides, amides, amines, phosphates and fluorides.

In an embodiment, the at least one base is selected from the group consisting of alkoxides, such as sodium tert-butoxide, potassium tert-butoxide and sodium methoxide, or from the group consisting of alkali metal amides, such as sodium amide and lithium diisopropylamide, alkali metal bis(trialkylsilyl)amides such as lithium bis-(trimethyl-silyl)amide and sodium bis-(trimethyl-silyl)amide, amines such as triethylamine, tributylamine, trimethylamine, diisopropyl ethyl amine, pyridine, N,N-dimethyl aminopyridine, 1,5-diazabicycl[4.3.0]nonene-5, 1,4-diazabicyclo[2.2.2]octane and 1,5-diazabicycl-[5.4.0]undecene-5 or from the group consisting of an alkali, alkaline earth carbonate, bicarbonate, hydroxide, phosphates and fluorides, such as sodium carbonate, sodium bicarbonate, sodium hydroxide, magnesium carbonate, magnesium bicarbonate, magnesium hydroxide, calcium carbonate, calcium bicarbonate, calcium hydroxide, barium carbonate, barium hydroxide, barium bicarbonate, potassium carbonate, potassium bicarbonate, potassium hydroxide, sodium acetate, potassium acetate, potassium phosphate, calcium acetate, cesium fluoride, potassium hydrogen phosphate, sodium phosphate, sodium hydrogen phosphate, sodium dihydrogen phosphate, potassium dihydrogen phosphate sodium trifluoroacetate, potassium trifluoroacetate, cesium carbonate, cesium bicarbonate and cesium hydroxide.

In a preferred embodiment, the at least one base is potassium hydroxide, sodium hydroxide, sodium tert-butoxide or potassium tert-butoxide.

In an embodiment, the at least one metal catalyst complex is selected from the group of transition metal catalysts including soluble or insoluble complexes of Platinum, Palladium, and Nickel. Nickel and Palladium are particularly preferred, and Palladium is most preferred.

In an embodiment, the at least one metal catalyst complex is selected from the group consisting of tetrakis(triphenylphosphine) palladium, dichlorobis(triphenylphosphine) palladium, tris(dibenzylideneacetone) dipalladium [Pd₂(dba)₃], bis(dibenzylideneacetone) dipalladium [Pd(dba)₂], palladium acetate, dichloro(1,5-cyclooctadiene) palladium and bis [cinnamyl palladium (II)] chloride, preferably bis [cinnamyl palladium (II)] chloride.

In yet another embodiment, the Buchwald-Hartwig reaction is carried out in the presence of the at least one metal catalyst complex and at least one phosphine ligand according to formula (I) wherein they are present in a molar ratio in the range of 1:1 to 1:10, preferably in the range of 1:2 to 1:6.

In a further embodiment, the at least one metal catalyst complex and the at least one phosphine ligand according to formula (I) are present in a molar ratio of 1:2, 1:3, 1:4, 1:5, or 1:6.

In a preferred embodiment, the Buchwald-Hartwig reaction is carried out at a temperature in the range of >80° C. to ≤140° C., more preferably at a temperature in the range of ≥90° C. to ≤120° C., even more preferably at a temperature in the range of ≥95° C. to ≤110° C.

In an embodiment, the Buchwald-Hartwig reaction is carried out at a pressure in the range of ≥1 bar to ≤30 bar, and more preferably at a pressure in the range of ≥1 to ≤10 bar.

In an embodiment, the Buchwald-Hartwig reaction is carried out in the presence of a ligand according to formula (I) wherein the amount of ligand is in the range of 0.001 mole % to 5 mol % based on the starting material used.

In an embodiment, the amount of ligand is in the range of 0.001 mol % to 4 mol %.

In an embodiment, the amount of ligand is in the range of 0.001 mol % to 3 mol %.

In an embodiment, the amount of ligand is in the range of 0.001 mol % to 2 mol %.

In an embodiment, the amount of ligand is in the range of 0.001 mol % to 1 mol %.

In an embodiment, the amount of ligand is in the range of 0.001 mol % to 0.5 mol %.

In an embodiment, the amount of ligand is in the range of 0.01 mol % to 0.5 mol %.

In an embodiment, the amount of ligand is in the range of 0.1 mol % to 0.5 mol %.

### EMBODIMENTS

In the following, there is provided a list of embodiments to further illustrate the present disclosure without intending to limit the disclosure to the specific embodiments listed below

1. A phosphine ligand of formula (I)

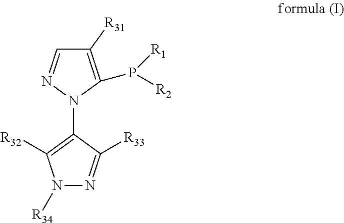

where,
R₁ and R₂ are, independently selected from substituted or unsubstituted C₃-C₁₄ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S,
R₃₁ is selected from substituted or unsubstituted, linear or branched C₁-C₆ alkyl, substituted or unsubstituted C₃-C₆ cycloalkyl, substituted or unsubstituted aryl and substituted or unsubstituted five or six membered heterocycloalkyl,
R₃₂, R₃₃, R₃₄, are each independently selected from substituted or unsubstituted, linear or branched C₁-C₆ alkyl, substituted or unsubstituted C₃-C₆ cycloalkyl, substituted or unsubstituted C₁-C₆ alkoxy, substituted or unsubstituted aryl, substituted or unsubstituted heteroaryl, substituted or unsubstituted N(R₁₂)₂, where R₁₂ is selected from substituted or unsubstituted C₁-C₆ alkyl, substituted or unsubstituted C₃-C₆ cycloalkyl, substituted or unsubstituted aryl, substituted or unsubstituted heteroaryl, substituted or unsubstituted heterocycloalkyl.

2. The phosphine ligand according to embodiments 1, wherein R₁ and R₂ are independently selected from cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, adamantyl, tetrahydronaphthyl, [3.3.0]bicyclo octane, [4.3.0]bicyclononane, [4.4.0]bicyclodecane and [2.2.2]bicyclooctane.

3. The phosphine ligand according to any of the preceding embodiments, wherein R₁ and R₂ are independently selected from adamantyl, cyclohexyl, cyclopentyl.

4. The phosphine ligand according to any of the preceding embodiments, wherein R₃₁ is in particular substituted or unsubstituted, linear or branched C₁-C₆ alkyl, in particular unsubstituted linear or branched C₁-C₆ alkyl.

5. The phosphine ligand according to embodiment 4, wherein R₃₁ is selected from methyl, ethyl, n-propyl, iso-propyl, n-butyl, iso-butyl, n-pentyl, iso-pentyl and n-hexyl.

6. The phosphine ligand according to embodiment 5, wherein R₃₁ is selected from methyl, ethyl, iso-propyl, n-propyl and n-butyl, preferably from methyl, iso propyl and n-propyl.

7. The phosphine ligand according to any of the preceding embodiments, wherein R₃₂, R₃₃, R₃₄, are, independently selected from substituted or unsubstituted straight chain C₁-C₆ alkyl, substituted or unsubstituted branched chain C₃-C₆ alkyl, substituted or unsubstituted C₃-C₆ cycloalkyl, substituted or unsubstituted C₁-C₆ alkoxy, substituted or unsubstituted aryl or substituted and unsubstituted heteroaryl.

8. The phosphine ligand according to embodiment 7, wherein R₃₂, R₃₃, R₃₄ are substituted or unsubstituted aryl.

9. The phosphine ligand according to embodiment 8, wherein R₃₂, R₃₃, R₃₄ are phenyl.

10. The phosphine ligand according to any of the preceding embodiments, wherein the complex of formula (I) is:

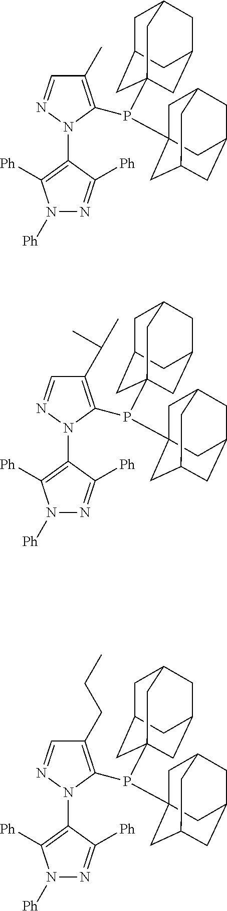 [SMILES: Cc1cnn(-c2c(-c3ccccc3)nn(-c3ccccc3)c2-c2ccccc2)c1P(C12CC3CC(CC(C3)C1)C2)C12CC3CC(CC(C3)C1)C2 ; CC(C)c1cnn(-c2c(-c3ccccc3)nn(-c3ccccc3)c2-c2ccccc2)c1P(C12CC3CC(CC(C3)C1)C2)C12CC3CC(CC(C3)C1)C2 ; CCCc1cnn(-c2c(-c3ccccc3)nn(-c3ccccc3)c2-c2ccccc2)c1P(C12CC3CC(CC(C3)C1)C2)C12CC3CC(CC(C3)C1)C2]

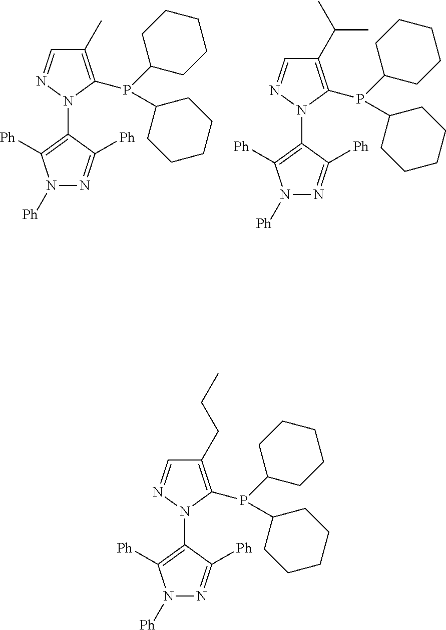

11. The phosphine ligand according to any of the preceding embodiments, wherein the complex of formula (I) is:

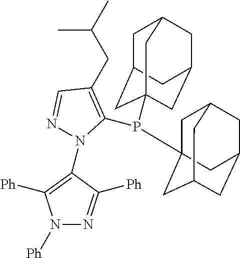 [SMILES: CC(C)Cc1cnn(-c2c(-c3ccccc3)nn(-c3ccccc3)c2-c2ccccc2)c1P(C12CC3CC(CC(C3)C1)C2)C12CC3CC(CC(C3)C1)C2]

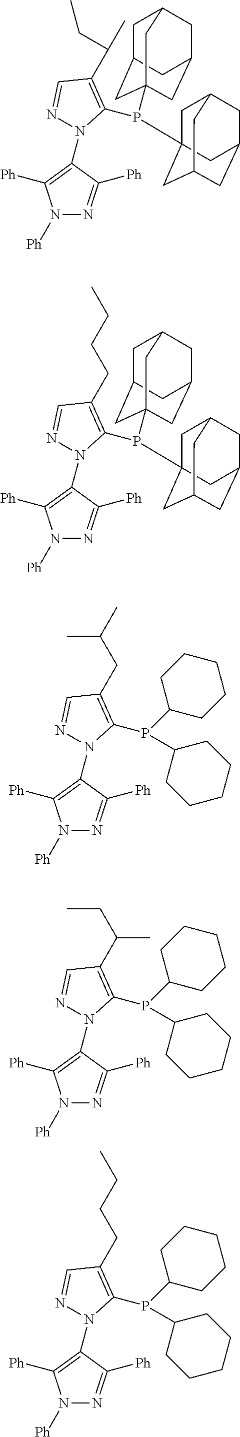 [SMILES: CCC(C)c1cnn(-c2c(-c3ccccc3)nn(-c3ccccc3)c2-c2ccccc2)c1P(C12CC3CC(CC(C3)C1)C2)C12CC3CC(CC(C3)C1)C2 ; CCCCc1cnn(-c2c(-c3ccccc3)nn(-c3ccccc3)c2-c2ccccc2)c1P(C12CC3CC(CC(C3)C1)C2)C12CC3CC(CC(C3)C1)C2 ; CC(C)Cc1cnn(-c2c(-c3ccccc3)nn(-c3ccccc3)c2-c2ccccc2)c1P(C1CCCCC1)C1CCCCC1 ; CCC(C)c1cnn(-c2c(-c3ccccc3)nn(-c3ccccc3)c2-c2ccccc2)c1P(C1CCCCC1)C1CCCCC1 ; CCCCc1cnn(-c2c(-c3ccccc3)nn(-c3ccccc3)c2-c2ccccc2)c1P(C1CCCCC1)C1CCCCC1]

12. A method of synthesizing the phosphine ligand of embodiment 1 comprising at least the steps of:
a) Lithiation of compound of formula (II)

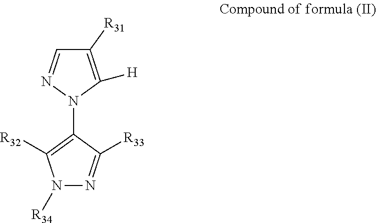

where,
R₃₁ is selected from H, substituted or unsubstituted, linear, or branched C₁-C₆ alkyl,
R₃₂, R₃₃, R₃₄, are each independently selected from substituted or unsubstituted, linear or branched C₁-C₆ alkyl, substituted or unsubstituted C₃-C₆ cycloalkyl, substituted or unsubstituted C₁-C₆ alkoxy, substituted or unsubstituted aryl, substituted or unsubstituted heteroaryl, substituted or unsubstituted N(R₁₂)₂, where R₁₂ is selected from substituted or unsubstituted C₁-C₆ alkyl, substituted or unsubstituted C₃-C₆ cycloalkyl, substituted or unsubstituted aryl, substituted or unsubstituted heteroaryl, substituted or unsubstituted heterocycloalkyl,
b) reacting the lithiated compound of formula (II) with compound of formula (III)

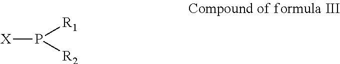

where X is Cl or Br,
R₁ and R₂ are independently selected from substituted or unsubstituted C₃-C₁₄ carbocycle, substituted or unsubstituted heterocycle comprising one or two 5- or 6-member rings and 1-4 heteroatoms selected from N, O and S, or substituted or unsubstituted heteroaryl comprising one or two 5- or 6-member rings and 1-4 heteroatoms selected from N, O and S,
to form phosphine ligand of formula (I) wherein the phosphine ligand of formula (I) is in a crude form,
c) optionally, purifying the phosphine ligand of formula (I) obtained in step b) by salt formation with an acid,
d) optionally, further purifying the phosphine ligand of formula (I) in step c) to give phosphine ligand of formula (I) in the pure form.

13. The process according to embodiment 12, wherein R₁ and R₂ are independently selected from adamantyl, cyclohexyl, or cyclopentyl.

14. The process according to any of the embodiments 12 or 13, wherein R₃₁ is in particular substituted or unsubstituted, linear or branched C₁-C₆ alkyl, in particular unsubstituted, linear or branched C₁-C₆ alkyl.

15. The process according to embodiment 14, wherein R₃₁ is selected from methyl, ethyl, n-propyl, iso-propyl, n-butyl, iso-butyl, n-pentyl, iso-pentyl and n-hexyl.

16. The process according to embodiment 15, wherein R₃₁ is selected from methyl, ethyl, iso-propyl, n-propyl and n-butyl, preferably from methyl, iso propyl and n-propyl.

17. The process according to any one of embodiments 12 to 16, wherein R₃₂, R₃₃, R₃₄, are, independently selected from substituted or unsubstituted straight chain C₁-C₆ alkyl, substituted or unsubstituted branched chain C₃-C₆ alkyl, substituted or unsubstituted C₃-C₆ cycloalkyl, substituted or unsubstituted C₁-C₆ alkoxy, substituted or unsubstituted aryl or substituted and unsubstituted heteroaryl.

18. The process according to embodiment 17, wherein R₃₂, R₃₃, R₃₄ are substituted or unsubstituted aryl.

19. The process according to embodiment 18, wherein R₃₂, R₃₃, R₃₄, are each phenyl.

20. The process according to any one of embodiments 12 to 19, wherein the lithiation is carried out in the presence of an organolithium reagent, which is in particular selected from methyl lithium, ethyl lithium, t-butyl lithium, n-butyl lithium, sec-butyl lithium, isopropyl lithium, lithium diisopropylamide or cyclohexanyllithium.

21. Use of the phosphine ligand according to any one of embodiments 1 to 11 and at least one transition metal catalyst to catalyze a reaction.

22. A method for catalyzing a reaction, comprising at least the step of adding a phosphine ligand according to any one of embodiments 1 to 11 and at least one transition metal.

23. The use or method according to any of the embodiments 21 or 22, wherein the transition metal is a transition metal of subgroup VIII of the Periodic Table of the Elements,

24. The use or method according to embodiment 23, wherein the transition metal is selected from palladium, nickel, platinum, rhodium, iridium, ruthenium and cobalt.

25. The use or method according to embodiment 24, wherein the transition metal is selected from palladium, nickel and platinum.

26. The use or method according to embodiment 24, wherein the transition metal comprises palladium.

27. The use or method according to any of the embodiments 21 or 26, wherein the reaction is selected from carbon-carbon coupling, carbon-heteroatom coupling, and polymerization reaction.

28. The use or method according to any of the embodiments 21 to 27, wherein the reaction is selected from the Heck reaction, Suzuki reaction, Sonogashira reaction, Negishi reaction, carbonyl alpha-arylation, allylic substitution, Buchwald-Hartwig carbon-nitrogen coupling, carbon-oxygen coupling or carbon-sulfur coupling.

29. The use or method according to embodiment 28, wherein the reaction is the Buchwald-Hartwig reaction to form aryl amines comprising at least the step of:
A) amination of a substituted haloaryl in the presence of
   i) at least one base,
   ii) at least one metal catalyst, in particular at least one transition metal catalyst,
   iii) at least one ligand of formula (I) according to any of the embodiments 1 to 10.

30. The use or method according to any one of embodiments 21 to 29, in particular according to embodiment 29, wherein the amount of ligand of formula (I) is in the range of 0.001 mol % to 5 mol % based on the starting material used.

### Examples

The presently claimed invention is illustrated in detail by non-restrictive working examples which follow.

### Materials

All reagents were purchased from commercial suppliers and used without further purification unless otherwise noted. Ligands of the invention were prepared according to procedure described herein. Potassium hydroxide was pulverized into a fine powder with a mortar and pestle and sifted through a 180 μm stainless-steel sieve to collect the smaller particles (<180 μm), which were stored in a nitrogen-filled glovebox and used. Anhydrous 1,4-dioxane were purchased and stored in a nitrogen-filled glovebox. All deuterated solvents were purchased from Cambridge Isotopes, freeze-pump-thawed three times, and stored over molecular sieves. All other solvents were dried by passing through activated alumina columns of solvent purification systems before use and stored in a nitrogen-filled glovebox.

Unless otherwise stated, all reactions were performed in flame-dried or oven-dried (minimum 3 hours at 165° C.) glassware in an N₂-atmosphere glovebox or with classical Schlenk technique. Dichloromethane (CH₂Cl₂), diethyl ether (Et₂O), toluene (PhMe), hexanes, and pentanes were obtained from a solvent dispensing system, having been previously degassed and passed through activated alumina columns. Dry, degassed tetrahydrofuran (THF) was obtained by storing degassed THF dispensed through an activated alumina column over 4 Å molecular sieves for 72 hours. Deuterated solvents were degassed by sparging with N₂ and dried over 3 Å molecular sieves for 72 hours. Volatile solvents were removed under reduced pressure with a rotary evaporator.

### Methods

Thin layer chromatography (TLC) was performed with Merck Kieselgel 60 F 254 fluorescent treated silica that were visualized by exposure to UV light (254 nm) and/or stained by a ceric ammonium molybdate solution.
   For the preparation of intermediates the following protocol was used:
   Thin layer chromatography (TLC) on Supelco® glass backed TLC plates (60 Å porosity, F-254 indicator) and visualized by UV irradiation and, when applicable, KMnO₄ stain solution, 12/SiO₂, ninhydrin stain solution, vanillin stain solution, or DNP stain solution.
Flash column chromatography was conducted with Fisher silica gel Grade 60 (230-400 mesh) and/or a Teledyne Isco Combiflash R f system with various sizes of RediSep R f gold columns.
   For the preparation of intermediates the following protocol was used:
   All column chromatography was performed using 230-400 mesh silica gel (40-63 μm), Brockman Activity I 60-325 mesh basic alumina gel, or Brockman Activity I (40-300 μm, 60 Å) neutral alumina gel; fractions were spotted using thin layer chromatography (TLC).
NMR spectra were recorded on Bruker AV- and NEO spectrometers operating at 300, 400, 500, and 600 MHz at the University of California, Berkeley.
   Chemical shifts (δ) in ¹H NMR spectra are reported in parts per million (ppm) relative to residual protonated solvent as a reference (o 7.26 for CDCl₃, δ 7.16 for C₆D₆, and δ 5.32 for CD₂Cl₂. Data for ¹H NMR spectroscopy are reported as follows: chemical shift, multiplicity (s=singlet, d=doublet, t=triplet, q=quartet, m=multiplet), coupling constant in Hertz (Hz) and integration.
   Chemical shifts in ¹³C NMR spectra are reported in ppm from the central peak of solvent (δ 77.16 for CDCl₃, δ 128.06 for C₆D₆, and δ 53.84 for CD₂Cl₂), and chemical shifts in ¹⁹F NMR spectra are reported in ppm relative to a CFCI3 external standard.

In the context of the intermediates ¹H NMR, ¹³C NMR, ¹⁹F NMR, and ³¹P NMR were recorded with Bruker AV, AVB, AVQ, Neo, and JEOL spectrometers operating at 400, 500, and 600 MHz for 1H (100, 125, 150 MHz for ¹³C; 376, 470, 564 MHz for ¹⁹F; 162, 202, 243 MHz for ³¹P). Chemical shifts are reported relative to the residual solvent signal per J. Org. Chem. 1997, 62, 7513-7514: ¹H NMR: 0=7.26 (CDCl₃), δ 5.32 (CD₂Cl₂), δ 1.94 (CD₃CN), δ 2.50 ((CD₃)₂SO), δ 7.16 (C₆D₆), δ 3.58, 1.73 (THF-d₈); ¹³C NMR: δ=77.16 (CDCl₃), δ 54.00 (CD₂Cl₂), δ 1.32, 118.26 (CD₃CN), δ 39.52 ((CD₃)₂SO), δ 128.06 (C₆D₆), δ 67.57, 25.37 (THF-d₈). NMR spectroscopy data are reported as follows: chemical shift (multiplicity, coupling constants where applicable (Hz), number of hydrogens). Splitting is reported with the following symbols: s=singlet, bs=broad singlet, d=doublet, t=triplet, dd=doublet of doublets, ddd=doublet of doublet of doublets, dt=doublet of triplets, tt=triplets of triplets, qr.=quartet, qn.=quintet, sx.=sextet, hept.=heptet, m=multiplet; app. denotes apparent splitting patterns.
Fourier-transform infrared (FTIR) spectra were taken on a Bruker Vertex 80 spectrometer (diamond anvil) with attenuated total reflection; only selected resonances are reported.
High-resolution mass spectroscopy (HRMS) was performed on a Perkin-Elmer AxION TOF MS or at the QB3/Chemistry Mass Spectrometry Facility at the University of California-Berkeley. IR and HRMS instruments are located at the Lawrence Berkeley National Laboratory Catalysis Center at University of California-Berkeley.
X-ray diffraction analysis was performed at the Small Molecule X-ray Crystallography Facility (CheXray) at the University of California-Berkeley.
Abbreviations used are: h for hour(s), min for minute(s), rt for retention time and ambient temperature for 20-25° C.
In formulae Ph refers to phenyl and Ad refers to adamantyl.
NMP refers to N-methyl pyrrolidone, MeOH refers to methanol and AcOH refers to acetic acid.

### Preparation of Intermediates

### Preparation Example 1: 4-Methyl-1′,3′,5′-triphenyl-1′H-1,4′-bipyrazole [4-Me-Bippy]

To a 100 mL round-bottom flask with a Teflon-coated stir bar was added 1,3-diphenyl-1,3-propanedione (3.12 g, 13.9 mmol, 1 equiv.), N-bromosuccinimide (3.20 g, 18.0 mmol, 1.30 equiv.), and N-methyl-2-pyrrolidone (12.0 mL). The reaction was stirred a room temperature for 18 h., then 4-methylpyrazole (3.76 mL, 48.7 mmol, 3.50 equiv.) was added. The reaction was warmed to 55° C. and stirred a further 24 h., then cooled to room temperature. The reaction mixture was diluted with glacial AcOH (9.00 mL) and a solution of phenylhydrazine (2.04 mL, 20.7 mmol, 1.50 equiv.) in MeOH (9.00 mL) was added. The reaction was stirred at room temperature for 24 h. over which time a white precipitate formed in the reaction flask. The formed precipitate was filtered off on a 10-15 μm porosity glass fritted funnel and washed with H₂O (15 mL). The filtercake was charged to a 50 mL round-bottom flask containing a Teflon-coated stir bar and H₂O (15 mL) and MeOH (15 mL) were added. The slurry was stirred at room temperature for 1 h. to granulate the product. The precipitate was filtered off on a 10-15 μm porosity glass fritted funnel and washed with H₂O (10 mL) and hexanes (10 mL). The filtercake was charged to a 250 mL round-bottom flask with a Teflon-coated stir bar and toluene (28 mL) was added. The solution was heated to 95° C. and stirred vigorously until all of the solid had dissolved. Heptane (70 mL) was added and the reaction was allowed to cool to room temperature with vigorous stirring, and once at room temperature the reaction was allowed to stir a further 1 h. The formed precipitate was collected by vacuum filtration on a 10-15 μm porosity glass fritted funnel, washed with heptane (3×10 mL), and dried under vacuum at 55° C. overnight to give a white solid (2.76 g, 52.7% yield).

¹H NMR (400 MHz, CDCl₃): δ 7.59 (s, 1H), 7.50 (m, 2H), 7.44-7.16 (m, 13H), 7.14 (s, 1H), 2.08 (s, 3H).

¹³C {¹H} NMR (101 MHz, CDCl₃): δ 148.0, 141.8, 140.9, 140.0, 131.4, 131.2, 129.4, 129.1, 129.0, 128.6, 128.5, 128.4, 127.9, 127.8, 126.9, 125.4, 121.4, 117.3, 9.0.

HRMS (ESI-TOF): Calc'd for [C₂₅H₂₁N₄]⁺ (M+H)⁺: m/z 377.1761, found 377.1773.

### Preparation Example 2: 4-Fluoro-1′,3′,5′-triphenyl-1′H-1,4′-bipyrazole [4-F-Bippy]

To a 250 mL round-bottom flask with a Teflon-coated stir bar was added 1,3-diphenyl-1,3-propanedione (11.2 g, 49.8 mmol, 1 equiv.), N-bromosuccinimide (11.5 g, 64.7 mmol, 1.30 equiv.), and NMP (43.0 mL). The reaction was stirred at room temperature for 18 h., then 4-fluoropyrazole (15.0 g, 174 mmol, 3.50 equiv.) was added in one portion. The reaction was warmed to 55° C. and stirred a further 24 h., then cooled to room temperature. The reaction mixture was diluted with glacial AcOH (32.0 mL) and a solution of phenylhydrazine (7.36 mL, 74.7 mmol, 1.50 equiv.) in MeOH (32.0 mL) was added. The reaction was stirred at room temperature for 24 h. over which time a precipitate formed in the reaction flask. The formed precipitate was filtered off on a 10-15 μm porosity glass fritted funnel and washed with H₂O (2×10 mL) and MeOH (3×10 mL). The filtercake was charged to a 250 mL round-bottom flask with a Teflon-coated stir bar and a solution of MeOH:H₂O (2:1, 60 mL) was added. The slurry was stirred at room temperature for 1 h. to granulate the product. The precipitate was filtered off onto a 10-15 μm porosity glass fritted funnel and washed with MeOH (2×10 mL) and hexanes (2×10 mL). The collected solid was dried under vacuum to give a white solid (11.3 g, 59.7% yield).

¹H NMR (400 MHz, CDCl₃): δ 7.61 (m, 1H), 7.48 (m, 2H), 7.40-7.21 (m, 12H), 7.15 (m, 2H) ¹⁹F {¹H} NMR (376 MHz, CDCl₃): δ −175.5.

¹³C {¹H} NMR (101 MHz, CDCl₃): δ 150.2 (d, J_{C-F}=248.2 Hz), 148.0, 141.2, 139.7, 130.8, 129.4, 129.3, 129.2, 128.73, 128.71 (d, J_{C-F}=12.9 Hz), 128.7, 128.0, 127.4, 126.9, 125.4, 121.2, 118.8 (d, J_{C-F}=26.4 Hz).

¹³C {¹H, ¹⁹F} NMR (101 MHz, CDCl₃): δ 150.2, 148.0, 141.2, 139.7, 130.8, 129.4, 129.3, 129.2, 128.71, 128.68, 128.1, 127.4, 126.9, 125.4, 121.2, 118.8.

HRMS (ESI-TOF): Calc'd for [C₂₄H₁₈FN₄]⁺ (M+H)⁺: m/z 381.1510, found 381.1499.

### Preparation Example 3: 4-Chloro-1′,3′,5′-triphenyl-1′H-1,4′-bipyrazole [4-CI-Bippy]

To a 250 mL round-bottom flask with a Teflon-coated stir bar was added 1,3-diphenyl-1,3-propanedione (4.48 g, 20.0 mmol, 1 equiv.), N-bromosuccinimide (4.63 g, 26.0 mmol, 1.30 equiv.), and NMP (20.0 mL). The reaction was stirred at room temperature for 18 h., then 4-chloropyrazole (7.18 g, 70.0 mmol, 3.50 equiv.) was added in one portion. The reaction was warmed to 55° C. and stirred a further 24 h., then cooled to room temperature. The reaction mixture was diluted with glacial AcOH (15.0 mL) and a solution of phenylhydrazine (2.95 mL, 30.0 mmol, 1.50 equiv.) in MeOH (15.0 mL) was added. The reaction was stirred at room temperature for 24 h. over which time a precipitate formed in the reaction flask. The formed precipitate was filtered off on a 10-15 μm porosity glass fritted funnel and washed with MeOH (50 mL). The filtercake was charged to a 500 mL round-bottom flask with a Teflon-coated stir bar and a solution of MeOH:H₂O (10:1, 400 mL) was added. The slurry was stirred at room temperature for 1 h. to granulate the product. The precipitate was filtered off onto a 10-15 μm porosity glass fritted funnel and washed with MeOH (3×50 mL) and pentane (3×50 mL). The collected solid was dried under vacuum to give 4-Chloro-1′,3′,5′-Triphenyl-1′H-1,4′-Bipyrazole [4-CI-Bippy] as an off-white solid (2.187 g, 27.6% yield).

¹H NMR (400 MHz, CDCl₃): δ 7.65 (s, 1H), 7.43 (dd, J=6.5, 3.0 Hz, 2H), 7.37-7.17 (m, 12H), 7.11 (app. d, J=7.2 Hz, 2H).

¹³C {¹H} NMR (101 MHz, CDCl₃): δ 147.9, 141.2, 139.7, 130.78, 130.76, 129.41, 129.35, 129.2, 128.8, 128.7, 128.1, 127.4, 127.0, 125.4, 120.7, 111.4.

HRMS (ESI-TOF): Calc'd for [C₂₄H₁₈ClN₄]⁺ (M+H)⁺: m/z 397.1214, found 397.1231.

### Preparation Example 4: 4-Bromo-1′,3′,5′-triphenyl-1′H-1,4′-bipyrazole [4-Br-Bippy]

To a 250 mL round-bottom flask with a Teflon-coated stir bar was added 1,3-diphenyl-1,3-propanedione (8.97 g, 40.0 mmol, 1 equiv.), N-bromosuccinimide (9.25 g, 52.0 mmol, 1.30 equiv.), and NMP (46.0 mL). The reaction was stirred at room temperature for 18 h., then 4-bromopyrazole (20.6 g, 140. mmol, 3.50 equiv.) was added in one portion. The reaction was warmed to 55° C. and stirred a further 24 h., then cooled to room temperature. The reaction mixture was diluted with glacial AcOH (35.0 mL) and a solution of phenylhydrazine (5.91 mL, 60.0 mmol, 1.50 equiv.) in MeOH (35.0 mL) was added. The reaction was stirred at room temperature for 24 h. over which time a precipitate formed in the reaction flask. The formed precipitate was filtered off on a 10-15 μm porosity glass fritted funnel and washed with MeOH (50 mL). The filtercake was charged to a 500 mL round-bottom flask with a Teflon-coated stir bar and a solution of MeOH:H₂O (4:1, 400 mL) was added. The slurry was stirred at room temperature for 1 h. to granulate the product. The precipitate was filtered off onto a 10-15 μm porosity glass fritted funnel and washed with MeOH (3×50 mL). The filtercake was charged to a 250 mL round-bottom flask with a Teflon-coated stir bar and toluene (75 mL) was added. The solution was heated to 95° C. and stirred vigorously until all of the solid had dissolved. Heptane (100 mL) was added and the reaction was allowed to cool to room temperature with vigorous stirring, and once at room temperature the reaction was allowed to stir a further 1 h. The formed precipitate was collected by vacuum filtration on a 10-15 μm porosity glass fritted funnel, washed with pentane (3×25 mL), and dried under vacuum to give a white solid (6.37 g, 36.1% yield).

HRMS (ESI-TOF): Calc'd for [C₂₄H₁₈BrN₄]⁺ (M+H)⁺: m/z 441.0709,

### Preparation Example 5: 4-Iodo-1′,3′,5′-triphenyl-1′H-1,4′-bipyrazole [4-I-Bippy]

To a 500 mL round-bottom flask with a Teflon-coated stir bar was added 1,3-diphenyl-1,3-propanedione (22.4 g, 100. mmol, 1 equiv.), N-bromosuccinimide (23.1 g, 130. mmol, 1.30 equiv.), and NMP (92.0 mL). The reaction was stirred at room temperature for 18 h., then 4-iodopyrazole (67.9 g, 350. mmol, 3.5 equiv.) was added in one portion. The reaction was warmed to 55° C. and stirred a further 24 h., then cooled to room temperature. The reaction mixture was diluted with glacial AcOH (70.0 mL) and a solution of phenylhydrazine (14.8 mL, 150. mmol, 1.50 equiv.) in MeOH (70.0 mL) was added. The reaction was stirred at room temperature for 24 h. over which time a precipitate formed in the reaction flask. The formed precipitate was filtered off on a 10-15 μm porosity glass fritted funnel and washed with MeOH (50 mL). The filtercake was charged to a 500 mL round-bottom flask with a Teflon-coated stir bar and a solution of MeOH:H₂O (10:1, 400 mL) was added. The slurry was stirred at room temperature for 1 h. to granulate the product. The precipitate was filtered off onto a 10-15 μm porosity glass fritted funnel and washed with MeOH (3×50 mL) and pentane (3×100 mL). The collected solid was dried under vacuum to give a fluffy, white solid (22.5 g, 46.1% yield).

¹H NMR (400 MHz, CDCl₃): δ 7.73 (s, 1H), 7.41 (app. dd, J=6.5, 3.0 Hz, 2H), 7.37-7.28 (m, 10H), 7.26-7.18 (m, 3H), 2.17 (m, 2H).

¹³C {¹H} NMR (101 MHz, CDCl₃): δ 147.9, 146.2, 141.0, 139.7, 137.2, 130.8, 129.4, 129.3, 129.2, 128.78, 128.76, 128.72, 128.1, 127.4, 127.0, 125.4, 120.4.

HRMS (ESI-TOF): Calc'd for [C₂₄H₁₈IN₄]⁺ (M+H)⁺: m/z 489.0571, found 489.0550.

### Preparation Example 6: 4-n-Butyl-1′,3′,5′-triphenyl-1′H-1,4′-bipyrazole [4-n-Bu-Bippy]

To a 250 mL round-bottom flask with a Teflon-coated stir bar was added under N₂: 4-iodo-1′,3′,5′-triphenyl-1′H,4′-bipyrazole [4-I-Bippy] (2.44 g, 5.00 mmol, 1 equiv.), CPhos [CAS No. 1160556-64-8] (0.0437 g, 0.100 mmol, 0.0200 equiv.), Pd-G4-Dimer [CAS No. 1581285-85-9] (0.0192 g, 0.0250 mmol, 0.00500 equiv.), and anhydrous THF (40.0 mL). The flask was cooled to 0° C. with an ice/water bath and a solution of (n-butyl) ZnCl [prepared separately by addition of n-butyllithium (2.50 M, 2.50 mL, 6.25 mmol, 1.25 equiv.) to a solution of anhydrous ZnCl₂ (0.886 g, 6.50 mmol, 1.30 equiv.) in THF (20.0 mL) at room temperature; the solution was stirred a further 30 min. at room temperature after addition of n-butyllithium was completed] was added slowly. The reaction mixture was warmed to room temperature and stirred for 7 h. at room temperature. The reaction was quenched with H₂O (20.0 mL) and extracted with EtOAc (3×25 mL). The combined organic layers were dried over anhydrous Na₂SO₄, filtered, and concentrated under reduced pressure. The residue was purified by flash column chromatography (SiO₂, 0→20% v/v Et₂O/pentane, R_{f}=0.3, visualised with UV light) to give 4-n-butyl-1′,3′,5′-triphenyl-1′H-1,4′-bipyrazole [4-n-Bu-Bippy] as a white solid (1.61 g, 77.0% yield).

¹H NMR (400 MHz, CDCl₃): δ 7.50 (s, 1H), 7.37 (app. dd, J=6.3, 2.7 Hz, 2H), 7.32-7.27 (m, 4H), 7.25-7.10 (m, 8H), 7.06 (m, 2H), 7.02 (s, 1H), 2.35 (t, J=7.4 Hz, 2H), 1.41 (qn., J=7.4 Hz, 2H), 1.19 (sx., J=7.2 Hz, 2H), 0.80 (t, J=7.3 Hz, 3H).

¹³C {¹H} NMR (101 MHz, CDCl₃): δ 148.0, 141.0, 140.9, 140.0, 131.2, 131.0, 129.4, 129.2, 128.9, 128.6, 128.50, 128.46, 128.0, 127.8, 126.9, 125.4, 123.2, 121.5, 33.0, 23.8, 22.1, 13.9.

HRMS (ESI-TOF): Calc'd for [C₂₈H₂₇N₄]⁺ (M+H)⁺: m/z 419.2230, found 419.2237.

### Preparation Example 7: 4-iso-Butyl-1′,3′,5′-triphenyl-1′H-1,4′-bipyrazole [4-iso-Bu-Bippy]

To a 250 mL round-bottom flask with a Teflon-coated stir bar was added under N₂: 4-iodo-1′,3′,5′-triphenyl-1′H,4′-bipyrazole [4-I-Bippy] (2.44 g, 5.00 mmol, 1 equiv.), CPhos [CAS No. 1160556-64-8] (0.0437 g, 0.100 mmol, 0.0200 equiv.), Pd-G4-Dimer [CAS No. 1581285-85-9] (0.0192 g, 0.0250 mmol, 0.00500 equiv.), and anhydrous THF (40.0 mL). The flask was cooled to 0° C. with an ice/water bath and a solution of (iso-butyl) ZnCl [prepared separately by addition of iso-butylmagnesium bromide (1.90 M, 4.17 mL, 6.25 mmol, 1.25 equiv.) to a solution of anhydrous ZnCl₂ (0.886 g, 6.50 mmol, 1.30 equiv.) in THF (20.0 mL) at room temperature; the solution was stirred a further 30 min. at room temperature after addition of iso-butylmagnesium bromide was completed] was added slowly. The reaction mixture was warmed to room temperature and stirred for 18 h. at room temperature. The reaction was quenched with H₂O (20.0 mL) and extracted with CH₂Cl₂ (3× 50 mL). The combined organic layers were dried over anhydrous Na₂SO₄, filtered, and concentrated under reduced pressure. The residue was purified by flash column chromatography (SiO₂, 0→5%→10% v/v EtOAc/hexane, R_{f}=0.3, visualised with UV light) to give 4-iso-butyl-1′,3′,5′-triphenyl-1′H-1,4′-bipyrazole [4-iso-Bu-Bippy] as a white solid (1.05 g, 50.1% yield).

¹H NMR (400 MHz, CDCl₃): δ 7.47 (s, 1H), 7.38 (app. dd, J=6.7, 3.0 Hz, 2H), 7.33-7.10 (m, 12H), 7.09-7.04 (m, 2H), 7.02 (s, 1H), 2.23 (d, J=2.23, 2H), 1.63 (app. nonet, J=6.7 Hz, 1H), 0.76 (d, J=6.6 Hz, 6H).

¹³C {¹H} NMR (101 MHz, CDCl₃): δ 147.9, 141.6, 140.9, 140.0, 131.5, 131.1, 129.4, 129.2, 129.0, 128.6, 128.51, 128.48, 128.0, 127.8, 126.9, 125.4, 121.7, 121.5, 33.4, 29.7, 22.2.

HRMS (ESI-TOF): Calc'd for [C₂₈H₂₇N₄]⁺ (M+H)⁺: m/z 419.2230, found 419.2224.

### Preparation Example 8: 4-Phenyl-1′,3′,5′-triphenyl-1′H-1,4′-bipyrazole [4-Ph-Bippy]

To a 250 mL round-bottom flask with a Teflon-coated stir bar was added under N₂: 4-iodo-1′,3′,5′-triphenyl-1′H,4′-bipyrazole [4-I-Bippy] (2.44 g, 5.00 mmol, 1 equiv.), CPhos [CAS No. 1160556-64-8] (0.0437 g, 0.100 mmol, 0.0200 equiv.), Pd-G4-Dimer [CAS No. 1581285-85-9] (0.0192 g, 0.0250 mmol, 0.00500 equiv.), and anhydrous THF (40.0 mL). The flask was cooled to 0° C. with an ice/water bath and a solution of PhZnCl [prepared separately by addition of phenyllithium (1.90 M, 3.29 mL, 6.25 mmol, 1.25 equiv.) to a solution of anhydrous ZnCl₂ (0.886 g, 6.50 mmol, 1.30 equiv.) in THF (20.0 mL) at room temperature; the solution was stirred a further 30 min. at room temperature after addition of phenyllithium was completed] was added slowly. The reaction mixture was warmed to room temperature and stirred for 18 h. at room temperature. The reaction was quenched with H₂O (20.0 mL) and extracted with CH₂Cl₂ (3×50 mL). The combined organic layers were dried over anhydrous Na₂SO₄, filtered, and concentrated under reduced pressure. The residue was purified by flash column chromatography (SiO₂, CH₂Cl₂, R_{f}=0.4, visualised with UV light) to give 4-phenyl-1′,3′,5′-triphenyl-1′H-1,4′-bipyrazole [4-Ph-Bippy] as a white solid (1.30 g, 59.3% yield).

¹H NMR (400 MHz, CDCl₃): δ 8.01 (d, J=0.6 Hz, 1H), 7.57 (d, J=0.6 Hz, 1H), 7.52-7.44 (m, 2H), 7.44-7.24 (m, 12H), 7.23-7.12 (m, 6H).

¹³C {¹H} NMR (101 MHz, CDCl₃): δ 147.9, 141.0, 139.9, 138.7, 132.2, 131.0, 129.6, 129.4, 129.2, 129.0, 128.7, 128.66, 128.64, 128.1, 127.6, 127.0, 126.8, 125.7, 125.5, 124.2, 121.1. HRMS (ESI-TOF): Calc'd for [C₃₀H₂₃N₄]⁺ (M+H)⁺: m/z 439.1917, found 493.1924.

### Preparation Example 9: 4-iso-Propyl-1′,3′,5′-triphenyl-1′H-1,4′-bipyrazole [4-iso-Pr-Bippy]

To a 250 mL round-bottom flask with a Teflon-coated stir bar was added under N₂: 4-iodo-1′,3′,5′-triphenyl-1′H,4′-bipyrazole [4-I-Bippy] (4.88 g, 10.0 mmol, 1 equiv.), CPhos [CAS No. 1160556-64-8] (0.175 g, 0.400 mmol, 0.0400 equiv.), Pd-G4-Dimer [CAS No. 1581285-85-9] (0.0768 g, 0.100 mmol, 0.0100 equiv.), and anhydrous THF (80.0 mL). The flask was cooled to 0° C. with an ice/water bath and a solution of (iso-propyl) ZnCI [prepared separately by addition of iso-propylmagnesium chloride lithium chloride complex (TurboGrignard) (1.30 M, 9.62 mL, 12.5 mmol, 1.25 equiv.) to a solution of anhydrous ZnCl₂ (1.77 g, 13.0 mmol, 1.30 equiv.) in THF (20.0 mL) at room temperature; the solution was stirred a further 30 min. at room temperature after addition of iso-propylmagnesium chloride lithium chloride complex was completed] was added slowly. The reaction mixture was warmed to room temperature and stirred for 7 h. at room temperature. The reaction was quenched with H₂O (20.0 mL) and extracted with CH₂Cl₂ (3× 50 mL). The combined organic layers were dried over anhydrous Na₂SO₄, filtered, and concentrated under reduced pressure. The residue was purified by flash column chromatography (SiO₂, 0→10% Et₂O/hexanes v/v, R_{f}=0.1, visualised with UV light) to give 4-(isopropyl)-1′,3′,5′-triphenyl-1′H-1,4′-bipyrazole [4-iso-Pr-Bippy] as a white solid (1.492 g, 36.9% yield).

¹H NMR (400 MHz, CDCl₃): iso-Pr-Bippy δ 7.56 (s, 1H), 7.42-7.06 (Ar—H 15 H), 7.03 (s, 1H), 2.76 (hept., J=6.9 Hz, 1H), 1.10 (d, J=6.9 Hz, 6H).

n-Pr-Bippy δ 7.52 (s, 1H), 7.42-7.06 (Ar—H, 15H), 7.05 (s, 1H), 2.35 (t, J=7.3 Hz, 2H), 1.47 (sx., J=7.3 Hz, 2H), 0.81 (t, J=7.3 Hz, 3H).

¹³C {¹H} NMR (101 MHz, CDCl₃): As Observed δ 148.0, 141.0, 140.8, 140.0, 139.5, 131.2, 131.0, 130.6, 129.7, 129.5, 129.4, 129.2, 129.0, 128.59, 128.58, 128.5, 128.48, 128.47, 127.9, 127.8, 126.93, 126.91, 125.5, 123.0, 121.6, 121.5, 26.2 (n-Pr-Bippy), 24.4 (iso-Pr-Bippy), 24.05 (n-Pr-Bippy), 24.02 (iso-Pr-Bippy), 13.6 (n-Pr-Bippy).

### Preparation Example 10:4-(pentafluoroethyl)-1′,3′,5′-triphenyl-1′H-1,4′-bipyrazole [4-C₂F₅-Bippy]

To a 40 mL vial with a Teflon-coated stir bar was added under N₂: 4-iodo-1′,3′,5′-triphenyl-1′H-1,4′-bipyrazole [4-I-Bippy] (2.44 g, 5.00 mmol, 1 equiv.), (1,10-phenanthroline) CuC₂F₅ [see Angew. Chem. Int. Ed. 2011, 50, 3793] (2.72 g, 7.50 mmol, 1.50 equiv.) and DMF (20.0 mL). The vial was sealed under N₂, heated to 50° C. and stirred at 50° C. for 18 h., then cooled to room temperature. The reaction mixture was diluted with Et₂O (25 mL) and filtered through a pad of Celite. The Celite pad was rinsed with additional washings of Et₂O (4×25 mL) and the collected filtrate was concentrated under reduced pressure. The residue was purified by flash column chromatography (SiO₂, 5% v/v EtOAc/hexanes, R_{f}=0.3, visualised with UV light) to give 4-(perfluoroethyl)-1′,3′,5′-triphenyl-1′H-1,4′-bipyrazole [4-C₂F₅-Bippy] as a white solid (2.29 g, 95.2% yield).

¹H NMR (400 MHz, CDCl₃): δ 7.96 (s, 1H), 7.63 (s, 1H), 7.49-7.43 (m, 2H), 7.43-7.22 (m, 11H), 7.13 (app. dd, J=8.3, 1.2 Hz, 2H).

¹⁹F {¹H} NMR (376 MHz, CDCl₃): δ −85.8 (s, 3F), −108.8 (s, 2F).

¹³C {¹H} NMR (101 MHz, CDCl₃): As Observedo 147.7, 141.1, 139.6, 139.1 (app. dt, J_{C-F}=4.8, 2.4 Hz), 133.5 (app td, J_{C-F}=5.4, 1.6 Hz), 130.5, 129.5, 129.3, 129.2, 128.9, 128.83, 128.80, 128.3, 127.1, 126.9, 125.4, 120.1, 119.0 (d, J_{C-F}=285.0 Hz), 112.6 (t, J_{C-F}=29.0 Hz), 111.6 (dd, J_{C-F}=249.6, 209.8 Hz).

¹³C {¹H, ¹⁹F} NMR (101 MHz, CDCl₃): As Observedo 147.7, 141.1, 139.6, 139.1, 133.5, 130.5, 129.5, 129.3, 129.2, 128.9, 128.83, 128.80, 128.3, 127.1, 126.9, 125.4, 120.1, 119.0, 112.6, 111.6.

HRMS (ESI-TOF): Calc'd for [C₂₆H₁₈F₅N₄]⁺ (M+H)⁺: m/z 481.1446, found 481.1467.

### Preparation Example 11:4-(pyrrolidin-1-yl)-1′,3′,5′-triphenyl-1′H-1,4′-bipyrazole [4-Pyrrolidino-Bippy]

To a 20 mL glass vial with a Teflon-coated stir bar was added under N₂: 4-iodo-1′,3′,5′-triphenyl-1′H-1,4′-bipyrazole [4-I-Bippy] (2.44 g, 5.00 mmol, 1 equiv.), anhydrous K₃PO₄ (2.12 g, 10.0 mmol, 2.00 equiv.), pyrrolidine (1.67 mL, 20.0 mmol, 4.00 equiv.), CuI (0.0952 g, 0.500 mmol, 0.100 equiv.), 2-acetylcyclohexanone (130. μL, 1.00 mmol, 0.200 equiv.), and DMF (10.0 mL). The vial was sealed and heated at 80° C. and stirred at 80° C. for 18 h., then cooled to room temperature. The reaction mixture was filtered through a pad of SiO₂, eluting with excess CH₂Cl₂ (300 mL). The collected filtrate was concentrated under reduced pressure and the formed residue was purified by flash column chromatography (SiO₂, 0→20% EtOAc/hexanes v/v, R_{f}=0.4, visualised with UV light) to give 4-(pyrrolidin-1-yl)-1′,3′,5′-triphenyl-1′H-1,4′-bipyrazole [4-Pyrrolidino-Bippy] as an off-white solid (0.825 g, 38.2% yield).

¹H NMR (400 MHz, CDCl₃): δ 7.56-7.02 (m, 16H), 6.76 (s, 1H), 2.99 (s, 4H), 1.91 (s, 4H).

¹³C {¹H} NMR (101 MHz, CDCl₃): δ 148.0, 141.0, 140.0, 137.5, 131.2, 130.4, 129.9, 129.5, 129.1, 128.9, 128.6, 128.5, 128.4, 127.9, 127.0, 125.5, 116.9, 51.2, 24.8.

### Preparation of Ligands

### Example 1: General Procedure for Preparation of the Ligand

To an oven-dried, 250 ml round-bottom flask containing a solution of bipyrazole (1 equiv) in THF (0.1 M), and a stir bar was removed from the glovebox and cooled to −78° C. To the solution was added n-butyllithium (1.5 equiv) dropwise at −78° C., and then the resulting mixture was stirred at the same temperature for 1.5 h.

An oven-dried, 50 ml pear shaped flask was charged with a suspension of BrPAd₂ or CIPCy₂ (1.5 equiv) in THF (0.1 M for BrPAd₂ and 0.5 M for CIPCy₂), and a stir bar and was then removed from the glovebox. To the flask containing lithiated bipyrazole in a water/ice/NaCl bath was added the suspension of halo phosphine by a cannular. The resulting mixture was stirred in the same cooling bath for 2 h, allowed to warm to room temperature, and stirred for overnight. The reaction was quenched with 1 N aq. NaOH (10 ml), and the aqueous layer was extracted with diethyl ether (10 ml×2 times). The collected organic extracts were washed with water, dried over Na₂SO₄, filtered, and concentrated under reduced pressure. The crude mixture was purified by flash column chromatography to give the product.

TABLE 1
Preparation of ligands and their characterization

| Ligand | Yield | Characterization |
|---|---|---|
| 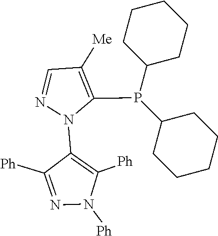 [SMILES: Cc1cnn(-c2c(-c3ccccc3)nn(-c3ccccc3)c2-c2ccccc2)c1P(C1CCCCC1)C1CCCCC1] | 51% | ¹H NMR (600 MHz, CDCl₃) δ 7.56 (s, 1H), 7.54-7.49 (m, 1H), 7.44-7.34 (m, 5H), 7.29-7.19 (m, 4H), 7.17-7.05 (m, 5H), 2.07 (s, 3H), 1.96-1.37 (m, 11H), 1.36- 1.01 (m, 11H). ³¹P NMR (243 MHz, CDCl₃) δ −12.36. |
| Cy-MeBippyPhos | | |
| 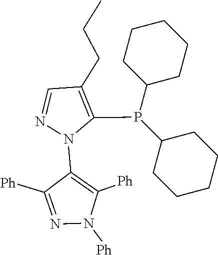 [SMILES: CCCc1cnn(-c2c(-c3ccccc3)nn(-c3ccccc3)c2-c2ccccc2)c1P(C1CCCCC1)C1CCCCC1] | 61% | ¹H NMR (500 MHz, CDCl₃) δ 7.58 (s, 1H), 7.56-7.49 (m, 1H), 7.41 (tdd, J= 9.9, 6.2, 2.9 Hz, 5H), 7.26-7.19 (m, 4H), 7.17- 7.03 (m, 5H), 2.43 (t, J = 7.3 Hz, 2H), 1.82- 1.59 (m, 11H), 1.55 (h, J = 7.4 Hz, 2H), 1.29-1.01 (m, 11H), 0.88 (t, J = 7.3 Hz, 3H). ³¹P NMR (202 MHz, CDCl₃) δ −12.41. |
| Cy-nPrBippyPhos | | |
| 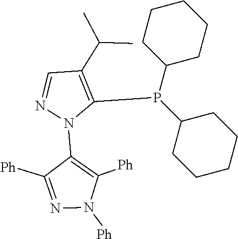 [SMILES: CC(C)c1cnn(-c2c(-c3ccccc3)nn(-c3ccccc3)c2-c2ccccc2)c1P(C1CCCCC1)C1CCCCC1] | − | ³¹P NMR (162 MHz, CDCl₃) δ −12.30 |
| Cy-iPrBippyPhos | | |
| 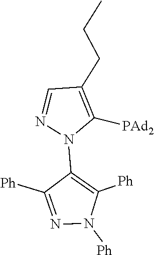 | 43% | ¹H NMR (500 MHz, CDCl₃) δ 7.87 (s, 1H), 7.50 (dd, J = 7.1, 1.6 Hz, 2H), 7.44 (dd, J = 7.7, 1.7 Hz, 2H), 7.33 (t, J = 7.5 Hz, 2H), 7.30-7.27 (m, 1H), 7.27-7.20 (m, 4H), 7.20-7.13 (m, 4H), 2.63 (hept, J = 7.4 Hz, 2H), 1.78 (h, J = 7.5 Hz, 2H), 1.54- 1.28 (m, 27H), 1.16 (d, J = 12.5 Hz, 3H), 1.04 (t, J = 7.3 Hz, 3H). ¹³C NMR (151 MHz, CDCl₃) δ 150.07, 141.30 (d, J = 2.6 Hz), 140.52, 140.40 (d, J = 2.4 Hz), 138.22 (d, J = 32.4 Hz), 132.79, 130.12, 129.29, 128.91, 128.79 (d, J = 6.3 Hz), 128.49 (d, J = 3.4 Hz), 128.40, 128.31, 128.00, 127.85, 127.48, 125.71, 122.31, 41.70 (dd, J = 45.2, 13.2 Hz), 36.93 (dd, J = 86.5, 22.0 Hz), 36.55 |
| Ad-nPrBippyPhos | | (d, J = 16.1 Hz), 28.86 (d, J = 9.3 Hz), |
| | | 14.45. |
| | | ³¹P NMR (202 MHz, CDCl₃) δ 16.05. |

### Example 2: Preparation of Ad-MeBippyPhos

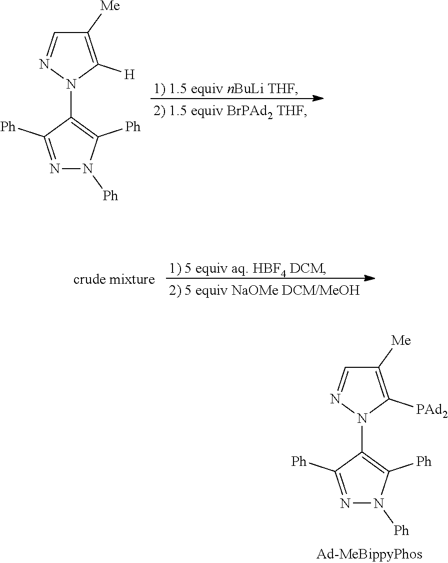

To an oven-dried, 250 ml round-bottom flask containing a solution of bipyrazole (1.53 g, 4.06 mmol, 1 equiv., OPRD 2008, 12, 480.) in THF (40.6 ml), and a stir bar was removed from the glovebox and cooled to −78° C. To the solution was added n-butyllithium (2.44 ml, 2.5 M in hexanes, 6.0 mmol, 1.5 equiv.) dropwise at −78° C., and then the resulting mixture was stirred at the same temperature for 1.5 h.

An oven-dried, 50 ml pear shaped flask was charged with a suspension of di(1-adamantyl) bromophosphine (2.32 g, 6.08 mmol, 1.5 equiv., Chem. Eur. J. 2013, 19, 17131.) in THF (20 ml), and a stir bar and was then removed from the glovebox. To the flask containing lithiated bipyrazole in a water/ice/NaCl bath was added the suspension of bromophosphine by a cannular. The pear-shaped flask was then washed with THF (5 ml×2 times) and then a solution was transferred to the round-bottom flask by a cannular. The resulting mixture was stirred in the same cooling bath for 2 h, allowed to warm to room temperature, and stirred for overnight (14 h). The reaction was quenched with 1 N aq. NaOH (30 ml), and the aqueous layer was extracted with diethyl ether (20 ml×2 times). The collected organic extracts were washed with water, dried over Na₂SO₄, filtered, and concentrated under reduced pressure. The crude mixture was used for the next step without further purification.

To a 50 ml round-bottom flask containing a solution of the crude mixture in DCM (20.3 ml, 0.2 M) and a stir bar was added HBF₄ (48 wt % aqueous solution, 2.65 ml, 20.3 mmol, 5 equiv) dropwise at 0° C. The resulting mixture was allowed to warm to room temperature and stirred for 1 h. Water (20 ml) was added to the reaction mixture, and the aqueous layer was extracted with DCM (15 ml) three times. The combined organic extracts were dried over MgSO₄, filtered, and concentrated under reduced pressure. The resulting solid was dissolved in the minimum amount of DCM and the solution was layered by diethyl ether to precipitate solids. The solid was collected by vacuum filtration on a fine fritted glass funnel and washed with diethyl ether (10 ml×5 times), and the collected solid was dried under vacuum overnight to provide the pure Ad-MeBippyPhos·HBF₄ as an orange solid (2.18 g, 2.85 mmol, 70% yield).

To a 50 ml round-bottom flask containing a solution of Ad-MeBippyPhos·HBF₄ (2.18 g, 2.85 mmol) in DCM (14.3 ml, 0.2 M) and a stir bar was added a solution of NaOMe (0.81 g, 95% purity, 14.2 mmol, 5 equiv.) in MeOH (3.1 ml) at 0° C. The resulting mixture was allowed to warm to room temperature and stirred for 4 h. Water (20 ml) was added to the resulting mixture, and the aqueous layer was extracted with DCM (15 ml) three times. The combined organic extracts were dried over Na₂SO₄, filtered, and concentrated under reduced pressure to afford pure Ad-MeBippyPhos as a yellow solid (1.87 g, 2.76 mmol, 68% yield for 4 steps).

¹H NMR (500 MHz, CDCl₃) δ 7.78 (s, 1H), 7.59-7.53 (m, 2H), 7.46 (d, J=7.9 Hz, 2H), 7.34 (td, J=8.2, 7.7, 2.0 Hz, 2H), 7.31-7.23 (m, 5H), 7.23-7.14 (m, 4H), 2.33 (s, 3H), 1.58-1.12 (m, 30H).

¹³C NMR (126 MHz, CDCl₃) δ 150.04 (d, J=2.0 Hz), 142.36 (d, J=2.5 Hz), 141.35 (d, J=2.8 Hz), 140.48, 139.02 (d, J=31.5 Hz), 132.84, 130.13, 129.30, 128.91, 128.48, 128.45, 128.35, 127.99, 127.89, 127.48, 125.69, 123.11 (d, J=6.3 Hz), 122.27, 41.53 (dd, J=34.1, 12.9 Hz), 37.20 (dd, J=95.6, 20.9 Hz), 36.53 (d, J=12.9 Hz), 28.81 (dd, J=9.2, 2.5 Hz), 14.22.

³¹P NMR (202 MHz, CDCl₃) δ 15.33.

### Example 3-6: Comparative Examples of Buchwald-Hartwig Reaction Using the Ligand as Per Example 1 or 2

### General Process Employed for the Buchwald-Hartwig Reaction

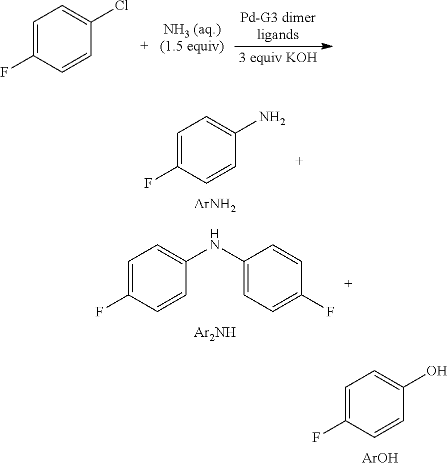

To an oven-dried, 4 ml vial charged with (2′-Amino-1,1′-biphenyl-2-yl) methanesulfonato palladium (II) dimer (0.5 mg, 97% purity, 0.5 μmol), ligand (2.0 μmol), and a PTFE-coated stir bar (12×4.5 mm) was added 1,4-dioxane (1.0 ml). The reaction mixture was stirred for 10 min to make a homogeneous solution. After aryl halide (0.1 mmol) and KOH (0.3 mmol) were added to the reaction mixture, the vial was sealed with a Teflon-lined cap, removed from the glovebox, and the reaction mixture was stirred at 25° C. for 10 min. Ammonium hydroxide solution (20 μl, 28% NH₃ basis, 0.3 mmol) was added to the reaction mixture and the mixture was heated at 100° C. for 24 h unless otherwise noted. The vial was allowed to cool to 25° C., and 1-fluoronaphtalene (0.1 mmol) or 1,3,5-trimethoxybenzene (0.1 mmol) was added as an internal standard, and the resulting mixture was transferred to an NMR tube through a syringe filter. Yields of the desired product and side products were determined by ¹H or ¹⁹F NMR spectroscopy. The reaction was performed using different ligands known in the art and the results are summarized in the below table 2.

TABLE 2
Comparative examples using Ligand as per example 1 and 2

| Example | | Product % conversion | | | | |
|---|---|---|---|---|---|---|
| no. | Ligand | ArCl | ArNH₂ | Ar₂NH | ArOH | Monoaryl:diaryl |
| 3* | tBuBippyPhos | — | 52 | 18 | — | 2.9:1 |
| 4* | AdBippyPhos | — | 69 | 12 | <1% | 6.0:1 |
| 5* | tBuBrettPhos | — | 61 | 2 | 25 | 42:1 |
| 6* | AdBrettPhos | — | 56 | 2 | 32 | 35:1 |
| 7* | MorDalPhos | 63 | — | 15 | — | n.d |
| 8 | Ad-Me BippyPhos | — | 91 | <1% | 1% | 178:1 |
| 9 | Ad-nPr BippyPhos | — | 91 | <1 | — | 233:1 |

*Examples 3- 7 were performed using known ligands (not as per the present invention) and Example 8 and 9 was performed using the ligand as per examples 1 or 2.

From table I it is evident that on using the phosphine ligand of formula (I) according to the present invention in catalyzing the Buchwald Hartwig reaction, higher percentage of the monoarylated product was formed with very less amount of hydroxylated product (impurity). Thus, very high selectivity (monoarylation selectivity) was seen in the reaction catalyzed by the phosphine ligand of formula (I) of the present invention.

### Examples 10 to 30: Buchwald-Hartwig Reaction Using the Ligand as Per Example 2 or 1 General Process Employed for Examples 10 to 30

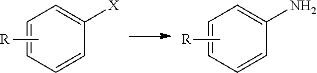

The aryl halide, the base, catalyst complex and ligand were mixed, and an amine (aq. Ammonia) was added to the glass vial and sealed. The mixture was heated for 100° C.-120° C. for 14-24 hours. The yields were determined ¹H NMR spectroscopy with 1,3,5-trimethoxybenzene or ¹⁹F NMR spectroscopy with 1-fluoronaphthalene as internal standards. The reaction conditions and the results are summarized in the below table 3

TABLE 3
Reaction conditions employed for examples 10 to 30

| Name | Reaction condition |
|---|---|
| R-1 | Aryl halide: 0.10 mmol, Aq ammonia: 0.30 mmol, KOH: 0.30 |
| | mmol in dioxane (1.0 ml), Pd-precatalyst: 0.0005 mmol, |
| | Ad-MeBippyPhos (0.0020 mmol) at 100° C. for 24 hours. |
| R-2 | Aryl halide: 0.10 mmol, Aq ammonia: 0.30 mmol, KOH: 0.30 |
| | mmol in dioxane (1.0 ml), Pd-precatalyst: 0.001 mmol, |
| | Ad-MeBippyPhos (0.0040 mmol) at 100° C. for 24 hours. |
| R-3 | Aryl halide: 0.10 mmol, Aq ammonia: 5 eq, KOH: 0.30 mmol in |
| | dioxane (1.0 ml), Pd-precatalyst: 0.0015 mmol, Ad-BippyPhos |
| | (0.0060 mmol) at 100° C. for 24 hours. |

TABLE 4
Summary of examples 10 to 30

| | Aryl halide 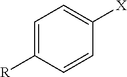 | | Reaction | Aryl amine 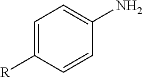 | | Ratio of Mono- |
|---|---|---|---|---|---|---|
| Ex no. | R | X | condition | R | Yield | aryl:diaryl |
| 10 | —N(CH₃)₂ | Cl | R-1 | —N(CH₃)₂ | 87% | >20:1 |
| 11 | —O(CH₃) | Cl | R-1 | —O(CH₃) | 88% | >20:1 |
| 12 | -TMS | Cl | R-1 | -TMS | 93% | >20:1 |
| 13 | -TMS | Br | R-1 | -TMS | 90% | >20:1 |
| 14 | -nC₄H₉ | Cl | R-1 | -nC₄H₉ | 91% | >20:1 |
| 15 | —C₆H₅ | Cl | R-1 | —C₆H₅ | 92% | >20:1 |
| 16 | —F | Cl | R-1 | —F | 91% | >20:1 |
| 17 | —F | Br | R-1 | —F | 96% | >20:1 |
| 18 | —O(CF₃) | Cl | R-1 | —O(CF₃) | 86% | >20:1 |
| 19 | —CF₃ | Cl | R-1 | —CF₃ | 88% | >20:1 |
| 20 | —C(═O)-t-C₄H₉ | Cl | R-1 | —C(═O)-t-C₄H₉ | 88% | >20:1 |
| 21 | —C(═O)—CH₃ | Cl | R-2 | —C(═O)—CH₃ | 93% | >20:1 |
| 22 | —C(═O)—N(C₂H₅)₂ | Cl | R-2 | —C(═O)—N(C₂H₅)₂ | 92% | >20:1 |
| 23 | —C(—OH)—CH₃ | Cl | R-1 | —C(—OH)—CH₃ | 98% | >20:1 |
| 24 | —NH₂ | Cl | R-2 | —NH₂ | 93% | >20:1 |
| 25 | —CN | Cl | R-1 | —CN | 88% | >20:1 |
| 26 | —NO₂ | Cl | R-2 | —NO₂ | 88% | >20:1 |
| 27 | —C(═CH₂)—CH₃ | Cl | R-1 | —C(═CH₂)—CH₃ | 86% | >20:1 |
| 28 | —S(CH₃) | Cl | R-3 | —S(CH₃) | 87% | 20:1 |
| 29 | —S(O₂)—C₆H₅ | Cl | R-1 | —S(O₂)—C₆H₅ | 86% | >20:1 |
| 30 | —S(O₂)—N(CH₃)₂ | Br | R-1 | —S(O₂)—N(CH₃)2 | 79% | >20:1 |

### Examples 31 to 50: Buchwald-Hartwig Reaction Using the Ligand as Per Example 2 or 1 General Process Employed for Examples 31 to 50

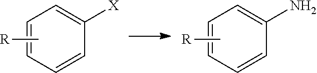

The aryl halide, the base, catalyst complex and ligand were mixed, and an amine (aq. Ammonia) was added to the glass vial and sealed. The mixture was heated for 100° C.-120° C. for 14-24 hours. The yields were determined ¹H NMR spectroscopy with 1,3,5-trimethoxybenzene or ¹⁹F NMR spectroscopy with 1-fluoronaphthalene as internal standards. The reaction conditions and the results are summarized in the below table 5.

TABLE 5
Reaction conditions employed for examples 31 to 49

| Name | Reaction condition |
|---|---|
| R-1 | Aryl halide: 0.10 mmol, Aq ammonia: 0.30 mmol, KOH: 0.30 |
| | mmol in dioxane (1.0 ml), Pd-precatalyst: 0.0005 mmol, |
| | Ad-MeBippyPhos (0.0020 mmol) at 100° C. for 24 hours. |
| R-2 | Aryl halide: 0.10 mmol, Aq ammonia: 0.30 mmol, KOH: 0.30 |
| | mmol in dioxane (1.0 ml), Pd-precatalyst: 0.001 mmol, |
| | Ad-MeBippyPhos (0.0040 mmol) at 100° C. for 24 hours. |
| R-3 | Aryl halide: 0.10 mmol, Aq ammonia 1.2 eq, KOH: 0.30 mmol in |
| | dioxane (1.0 ml), Pd-precatalyst: 0.001 mmol, Ad-MeBippyPhos |
| | (0.0040 mmol) at 100° C. for 24 hours. |
| R-4 | Aryl halide: 0.10 mmol, Aq ammonia: 0.30 mmol, KOH: 0.30 |
| | mmol in dioxane (1.0 ml), Pd-precatalyst: 0.001 mmol, |
| | Ad-MeBippyPhos (0.0040 mmol) at 100° C. for 48 hours. |
| R-5 | Aryl halide: 0.10 mmol, Aq ammonia: 5 eq, KOH: 0.30 mmol in |
| | dioxane (1.0 ml), Pd-precatalyst: 0.0005 mmol, Ad-BippyPhos |
| | (0.0020 mmol) at 50° C. for 24 hours |
| R-6 | Aryl halide: 0.10 mmol, Aq ammonia: 0.30 mmol, KOH: 0.30 |
| | mmol in dioxane (1.0 ml), Pd-precatalyst: 0.0025 mmol, |
| | Ad-MeBippyPhos (0.010 mmol) at 100° C. for 24 hours. |

TABLE 6
Summary of examples 31 to 49.

| | | | | | | Ratio of |
|---|---|---|---|---|---|---|
| Ex | Aryl halide | | Reaction | | | Mono- |
| no. | | X | condition | Aryl amine | Yield | aryl:diaryl |
| 31 | 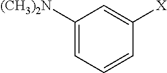 | Br | R-1 | 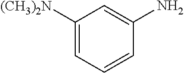 | 87% | >20:1 |
| 32 | 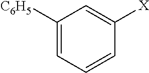 | Br | R-1 | 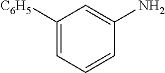 | 91% | >20:1 |
| 33 | 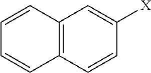 | Cl | R-1 | 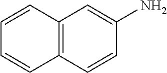 [SMILES: Nc1ccc2ccccc2c1] | 99% | >20:1 |
| 34 | 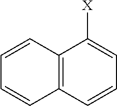 | Cl | R-5 | 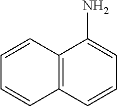 [SMILES: Nc1cccc2ccccc12] | 91% | >20:1 |
| 35 | 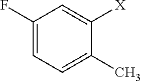 | Cl | R-5 | 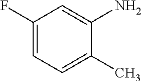 | 94% | >20:1 |
| 36 | 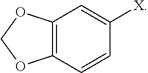 | Cl | R-1 | 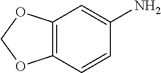 | 92% | >20:1 |
| 37 | 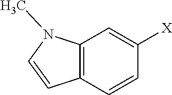 | Br | R-1 | 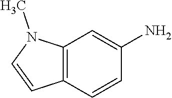 | 86% | >20:1 |
| 38 | 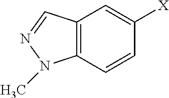 | Br | R-1 | 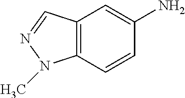 | 88% | >20:1 |
| 39 | 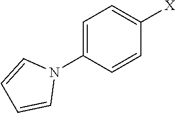 | Cl | R-1 | 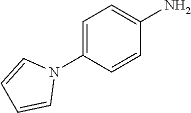 [SMILES: Nc1ccc(-n2cccc2)cc1] | 99% | >20:1 |
| 40 | 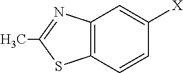 | Cl | R₆ | 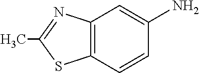 | 92% | >20:1 |
| 41 | 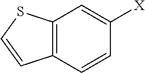 | Br | R-2 | 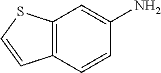 | 87% | >20:1 |
| 42 | 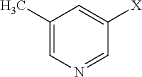 | Cl | R-1 | 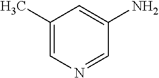 | 89% | >20:1 |
| 43 | 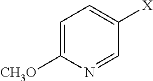 | Cl | R-1 | 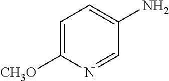 | 78% | >20:1 |
| 44 | 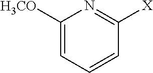 | Cl | R-3 | 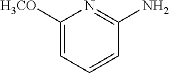 | 77% | >20:1 |
| 45 | 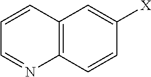 | Cl | R₁ | 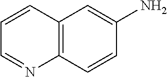 [SMILES: Nc1ccc2ncccc2c1] | 87% | >20:1 |
| 46 | 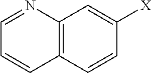 | Cl | R-1 | 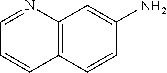 [SMILES: Nc1ccc2cccnc2c1] | 88% | >20:1 |
| 47 | 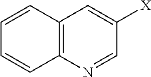 | C | R-1 | 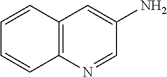 [SMILES: Nc1cnc2ccccc2c1] | 81% | >20:1 |
| 48 | 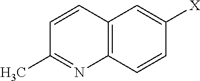 | Cl | R-1 | 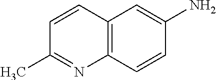 | 90% | 20:1 |
| 49 | 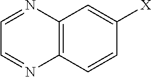 | Cl | R-4 | 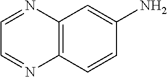 [SMILES: Nc1ccc2nccnc2c1] | 93% | >20:1 |

From the examples in table 4 and 6, it is evident that on using the phosphine ligand of formula (I) according to the present invention in catalyzing the Buchwald Hartwig reaction, higher percentage of the monoarylated product was formed. Thus, very high selectivity (monoarylation selectivity) was seen in the reaction catalyzed by the phosphine ligand of formula (I) of the present invention.

1. A phosphine ligand of formula (I) 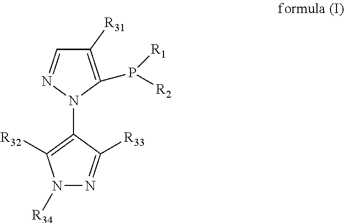
where,
R₁ and R₂ are independently selected from substituted or unsubstituted C₃-C₁₄ carbocycle, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S,
R₃₁ is selected from substituted or unsubstituted, linear or branched C₁-C₆ alkyl, substituted or unsubstituted C₃-C₆ cycloalkyl, substituted or unsubstituted aryl, and substituted or unsubstituted five or six membered heterocycloalkyl,
R₃₂, R₃₃, R₃₄ are each independently selected from substituted or unsubstituted, linear or branched C₁-C₆ alkyl, substituted or unsubstituted C₃-C₆ cycloalkyl, substituted or unsubstituted C₁-C₆ alkoxy, substituted or unsubstituted aryl, substituted or unsubstituted heteroaryl, or substituted or unsubstituted N(R₁₂)₂, where R₁₂ is selected from substituted or unsubstituted C₁-C₆ alkyl, substituted or unsubstituted C₃-C₆ cycloalkyl, substituted or unsubstituted aryl, substituted or unsubstituted heteroaryl, or substituted or unsubstituted heterocycloalkyl.

2. The phosphine ligand according to claim 1, wherein R₁ and R₂ are independently selected from cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, adamantyl, tetrahydronaphthyl, [3.3.0]bicyclo octane, [4.3.0]bicyclononane, [4.4.0]bicyclodecane, and [2.2.2]bicyclooctane.

3. The phosphine ligand according to claim 1, wherein R₁ and R₂ are independently selected from adamantyl, cyclohexyl, and cyclopentyl.

4. The phosphine ligand according to claim 1, wherein R₃₁ is unsubstituted, linear or branched C₁-C₆ alkyl.

5. The phosphine ligand according to claim 4, wherein R₃₁ is selected from methyl, ethyl, iso-propyl, n-propyl, and n-butyl.

6. The phosphine ligand according to claim 1, wherein R₃₂, R₃₃, R₃₄ are independently selected from substituted or unsubstituted straight chain C₁-C₆ alkyl, substituted or unsubstituted branched chain C₃-C₆ alkyl, substituted or unsubstituted C₃-C₆ cycloalkyl, substituted or unsubstituted C₁-C₆ alkoxy, substituted or unsubstituted aryl, or substituted and unsubstituted heteroaryl.

7. The phosphine ligand according to claim 6, wherein R₃₂, R₃₃, R₃₄ are phenyl.

8. The phosphine ligand according to claim 1, wherein the complex of formula (I) is selected from the group consisting of

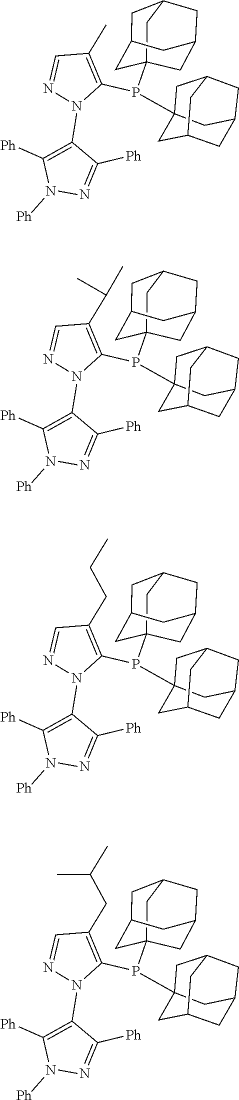 [SMILES: Cc1cnn(-c2c(-c3ccccc3)nn(-c3ccccc3)c2-c2ccccc2)c1P(C12CC3CC(CC(C3)C1)C2)C12CC3CC(CC(C3)C1)C2 ; CC(C)c1cnn(-c2c(-c3ccccc3)nn(-c3ccccc3)c2-c2ccccc2)c1P(C12CC3CC(CC(C3)C1)C2)C12CC3CC(CC(C3)C1)C2 ; CCCc1cnn(-c2c(-c3ccccc3)nn(-c3ccccc3)c2-c2ccccc2)c1P(C12CC3CC(CC(C3)C1)C2)C12CC3CC(CC(C3)C1)C2 ; CC(C)Cc1cnn(-c2c(-c3ccccc3)nn(-c3ccccc3)c2-c2ccccc2)c1P(C12CC3CC(CC(C3)C1)C2)C12CC3CC(CC(C3)C1)C2]

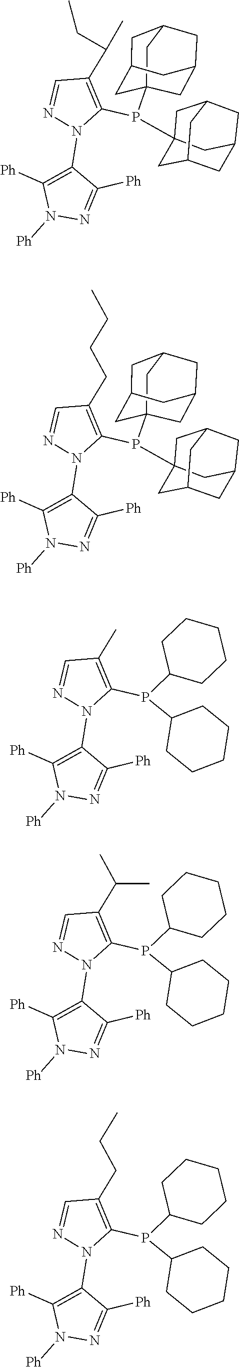 [SMILES: CCC(C)c1cnn(-c2c(-c3ccccc3)nn(-c3ccccc3)c2-c2ccccc2)c1P(C12CC3CC(CC(C3)C1)C2)C12CC3CC(CC(C3)C1)C2 ; CCCCc1cnn(-c2c(-c3ccccc3)nn(-c3ccccc3)c2-c2ccccc2)c1P(C12CC3CC(CC(C3)C1)C2)C12CC3CC(CC(C3)C1)C2 ; Cc1cnn(-c2c(-c3ccccc3)nn(-c3ccccc3)c2-c2ccccc2)c1P(C1CCCCC1)C1CCCCC1 ; CC(C)c1cnn(-c2c(-c3ccccc3)nn(-c3ccccc3)c2-c2ccccc2)c1P(C1CCCCC1)C1CCCCC1 ; CCCc1cnn(-c2c(-c3ccccc3)nn(-c3ccccc3)c2-c2ccccc2)c1P(C1CCCCC1)C1CCCCC1]

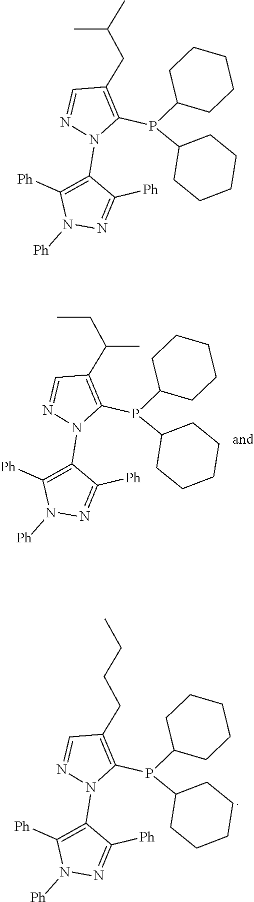 [SMILES: CC(C)Cc1cnn(-c2c(-c3ccccc3)nn(-c3ccccc3)c2-c2ccccc2)c1P(C1CCCCC1)C1CCCCC1 ; CCC(C)c1cnn(-c2c(-c3ccccc3)nn(-c3ccccc3)c2-c2ccccc2)c1P(C1CCCCC1)C1CCCCC1 ; CCCCc1cnn(-c2c(-c3ccccc3)nn(-c3ccccc3)c2-c2ccccc2)c1P(C1CCCCC1)C1CCCCC1]

9. A method of synthesizing the phosphine ligand of claim 1 comprising:
a) lithiation of a compound of formula (II) 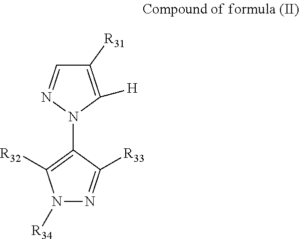
where,
R₃₁ is substituted or unsubstituted, linear or branched C₁-C₆ alkyl,
R₃₂, R₃₃, R₃₄, are each independently selected from substituted or unsubstituted, linear or branched C₁-C₆ alkyl, substituted or unsubstituted C₃-C₆ cycloalkyl, substituted or unsubstituted C₁-C₆ alkoxy, substituted or unsubstituted aryl, substituted or unsubstituted heteroaryl, or substituted or unsubstituted N(R₁₂)₂, where R₁₂ is selected from substituted or unsubstituted C₁-C₆ alkyl, substituted or unsubstituted C₃-C₆ cycloalkyl, substituted or unsubstituted aryl, substituted or unsubstituted heteroaryl, or substituted or unsubstituted heterocycloalkyl,
b) reacting the lithiated compound of formula (II) with a compound of formula (III) 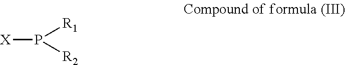
where X is Cl or Br,
R₁ and R₂ are independently selected from substituted or unsubstituted C₃-C₁₄ carbocycle, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S,
to form the phosphine ligand of formula (I), wherein the phosphine ligand of formula (I) is in a crude form,
c) optionally purifying the phosphine ligand of formula (I) obtained in step b).

10. The process according to claim 9, wherein R₁ and R₂ are independently selected from adamantyl, cyclohexyl, or cyclopentyl.

11. The process according to claim 9, wherein R₃₁ is unsubstituted, linear or branched C₁-C₆ alkyl.

12. The process according to claim 9, wherein R₃₂, R₃₃, R₃₄, are each phenyl.

13. The process according to claim 9, wherein the lithiation is carried out in the presence of an organolithium reagent selected from methyl lithium, ethyl lithium, t-butyl lithium, n-butyl lithium, sec-butyl lithium, iso-propyl lithium, lithium diisopropylamide, or cyclohexanyl lithium.

14. (canceled)

15. A method for catalyzing a reaction comprising adding a phosphine ligand according to claim 1 and at least one transition metal.

16. The method according to claim 15, wherein the reaction is selected from carbon-carbon coupling, carbon-heteroatom coupling, or a polymerization reaction.

17. The use er method according to claim 16, wherein the reaction is selected from Heck reaction, Suzuki reaction, Sonogashira reaction, Negishi reaction, ketone alpha-arylation, aldehyde alpha-arylation, allylic substitution, Buchwald-Hartwig reaction, carbon oxygen coupling, and carbon-sulfur coupling.

18. The method according to claim 17, wherein the reaction is the Buchwald-Hartwig reaction to form an aryl amine comprising:
A) amination of a substituted haloaryl in the presence of
i) at least one base,
ii) at least one metal catalyst,
iii) at least one ligand of formula (I).

19. The use er method according to claim 18, wherein an amount of the ligand of formula (I) is in a range of 0.001 mol % to 5 mol % based on the starting material used.
